SEQUENCE LISTING (5 sequences)

SEQ ID NO: 1 - RNA, 20 nt, synthetic construct
  source 1..20: mol_type = other RNA; note = Ptpn6-sgRNAI
caaacttctcccctccgtac

SEQ ID NO: 2 - RNA, 20 nt, synthetic construct
  source 1..20: mol_type = other RNA; note = Ptpn6-sgRNA2
gacttctatgacctgtacgg

SEQ ID NO: 3 - RNA, 20 nt, synthetic construct
  source 1..20: mol_type = other RNA; note = Ptpn6-sgRNA3
ctggcccaggttccccgctc

SEQ ID NO: 4 - RNA, 20 nt, synthetic construct
  source 1..20: mol_type = other RNA; note = Non-targeting sgRNA
gcgaggtattcggctccgcg

SEQ ID NO: 5 - protein, 8 aa, synthetic construct
  source 1..8: note = H-2Kb-SIINFEKL
SIINFEKL

(19) United States
(12) Patent Application Publication
(10) Pub. No.: US 2024/0424019 A1
(43) Pub. Date: Dec. 26, 2024

(54) SHP1 MODIFIED DENDRITIC CELLS AND EXTRACELLULAR VESICLES DERIVED THEREFROM AND ASSOCIATED METHODS

(71) Applicant: BOARD OF REGENTS, THE UNIVERSITY OF TEXAS SYSTEM, Austin, TX (US)

(72) Inventors: Dihua YU, Houston, TX (US); Xiangliang YUAN, Houston, TX (US); Yimin DUAN, Houston, TX (US); Yi XIAO, Houston, TX (US); Yutao QI, Houston, TX (US)

(21) Appl. No.: 18/699,447

(22) PCT Filed: Oct. 6, 2022
(86) PCT No.: PCT/US2022/077656

§ 371 Date: Apr. 8, 2024

Related U.S. Application Data
(60) Provisional application No. 63/252,721, filed on Oct. 6, 2021.

(51) Int. Cl. A61K 35/15; A61K 39/00; A61K 45/06; A61P 31/00; C12N 5/0784

(52) U.S. Cl. CPC A61K 35/15; A61K 39/0011; A61K 45/06; A61P 31/00; C12N 5/0639; A61K 2039/5154; A61K 2039/5156; C12N 2500/38

(57) ABSTRACT

Methods of activating immune response for treating cancer, infections, or other immune-deficient diseases in patients are provided. Also provided are compositions, formulations, and kits for use in such methods, which comprise dendritic cells (DCs), or extracellular vesicles derived therefrom (DC-EVs), in which src homologous region 2 domain-containing phosphatase-1 (SHIP1) expression and/or activity is inhibited and which have been loaded with disease-specific or disease-associated antigens or peptides. Such SHIP1-modified DC-EVs or DCs, or formulations thereof, may be administered to a patient as monotherapy, and/or as a component of combinational therapies with other therapies in the prevention and treatment of cancer and for boosting antigen-specific immune function in the treatment of other diseases. Also provided are methods of making such SHIP1-modified DC-EVs or DCs and formulations thereof.

### CROSS-REFERENCE TO RELATED APPLICATIONS

This application claims priority to and the benefit of U.S. Provisional Application No. 63/252,721. filed on Oct. 6. 2021, the entire content of which is herein incorporated by reference for all purposes.

### STATEMENT AS TO RIGHTS TO INVENTIONS MADE UNDER FEDERALLY SPONSORED RESEARCH AND DEVELOPMENT

This invention was made with government support under grant numbers R01 CA231149 and R01 CA208213 awarded by the National Institutes of Health. The government has certain rights in the invention.

### SEQUENCE LISTING

The official copy of the sequence listing is submitted electronically via Patent Center as an XML formatted sequence listing with a file named ______, created on ______, and having a size of ______, and is filed concurrently with the specification. The sequence listing contained in this xml formatted document is part of the specification and is herein incorporated by reference in its entirety.

### BACKGROUND

Cancer affects millions of people each year. While considerable progress in the treatment of cancer has been made over the last decades, better treatment is still in great need. Recently, the field of immune checkpoint therapies (ICT) has joined the ranks of surgery, radiation, chemotherapy, and targeted therapy as a pillar of cancer therapy. ICT targeting regulatory pathways in T cells, such as anti-cytotoxic lymphocyte antigen-4 (CTLA-4) and anti-programmed cell death protein 1 (PD-1)/programmed death-ligand 1 (PD-L1), have achieved striking clinical success in augmenting antitumor T-cell responses and providing long-lasting protection in a subset of cancer patients. However, ICT is still limited by low clinical response rates and some patients develop resistance to therapy after the initial response. Thus, maximizing the therapeutic effects of ICT is an unmet clinical challenge.

A successful anti-cancer immune response requires a series of stepwise events in the cancer-immunity cycle (Chen and Mellman, 2013) that are critically regulated and can be therapeutically modulated. A key determinant of an effective cancer immunotherapy response is immunogenic antigen presentation in tumors (Cruz et al., 2017; Wculek et al., 2020). Dendritic cells (DCs), as the sentinel antigen-presenting cells (APCs) of the immune system, play a central role in initiating antigen-specific immunity. The initiation and maintenance of adaptive T-cell antitumor immunity require multifaceted modes of communication between DCs and other immune cells, including direct intercellular contact, secreted soluble signaling molecules, and extracellular vesicles (EVs). Both DCs and DC-derived extracellular vesicles (DC-EVs) are able to stimulate antitumor immune responses.

There is thus a need for new, effective methods for enhancing immune responses using DCs or DC-EVs in patients, such as patients with cancer or an infection. The present disclosure satisfies this need and provides other advantages as well.

### BRIEF SUMMARY

In one aspect, the present disclosure provides a method of treating cancer or an infection in a subject, the method comprising administering to the subject dendritic cell (DC)-derived extracellular vesicles (DC-EVs), wherein the DC-EVs are obtained from dendritic cells that have been treated to inhibit src homologous region 2 domain-containing phosphatase-1 (SHP1) in the cells, and wherein the dendritic cells have been loaded with an antigen present in the cancer of the subject or in an infectious agent present in the subject.

In some embodiments, the dendritic cells are loaded by culturing them in the presence of the antigen for a duration sufficient to allow the presentation of the antigen on the surface of the dendritic cells, and wherein the dendritic cells are cultured under conditions permissible for exosome production. In some embodiments, the method further comprises administering the dendritic cells to the subject together with the DC-EVs. In some embodiments, the DC-EVs are harvested from the dendritic cells and concentrated prior to administration to the subject. In some embodiments, the dendritic cells are monocyte-derived dendritic cells (MoDCs) or induced pluripotent stem cell (iPSC)-derived dendritic cells (iPSC-DCs). In some embodiments, SHP1 is inhibited in the dendritic cells by contacting the cells with a small molecule SHP1 inhibitor, by silencing SHP1 in the cells, or by editing the SHP1 gene in the cells so as to reduce or eliminate its expression. In some embodiments, the small molecule SHP1 inhibitor is vitamin E (DL-α-tocopherol) or an analog or variant thereof.

In some embodiments, the small molecule SHP1 inhibitor comprises the structure

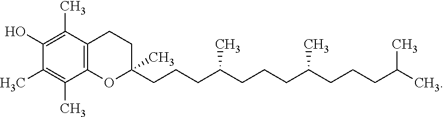 [SMILES: Cc1c(C)c2c(c(C)c1O)CC[C@@](C)(CCC[C@H](C)CCC[C@H](C)CCCC(C)C)O2]

In some embodiments, the small molecule SHP1 inhibitor is at least one of TPI-1, NSC-87877, suramin, or sodium stibogluconate (SSG). In some embodiments, the SHP1 gene is edited in the cells using a gene editing system. In some embodiments, the SHP1 gene is edited in the cells using a CRISPR-Cas system, comprising a guide RNA that targets the SHP1 locus.

In some embodiments of the provided methods, the antigen is: (a) a tumor antigen that is expressed on the surface of a tumor cell of the subject's cancer or an antigenically active fragment thereof, or a peptide fragment of the tumor antigen that is capable of being presented by dendritic cells to T cells: or (b) a viral or bacterial antigen that is expressed on the surface of the infectious agent causing the infection or an antigenically active fragment thereof, or a peptide fragment of the viral or bacterial antigen that is capable of being presented by dendritic cells to T cells. In some embodiments, the extracellular vesicles comprise exosomes. In some embodiments, inhibiting SHP1 in the dendritic cells and culturing the cells in the presence of the antigen results in an increased amount of the antigen on the surface of the dendritic cells as compared to control dendritic cells that have been cultured in the presence of the antigen but in which SHP1 has not been inhibited. In some embodiments, the extracellular vesicles harvested from the dendritic cells have an increased amount of the antigen on their surfaces as compared to extracellular vesicles harvested from the control dendritic cells that have been cultured in the presence of the antigen but in which SHP1 has not been inhibited. In some embodiments, the subject has cancer and the method further comprises administering an anti-cancer therapy to the subject as combinational therapy, wherein the anti-cancer therapy comprises surgical therapy, chemotherapy, radiation therapy, cryotherapy, hormonal therapy, and/or immunotherapy.

In some embodiments, the anti-cancer therapy comprises administering a tyrosine kinase inhibitor, co-stimulatory mAb, epigenetic modulator, chemotherapeutic agent, radiation therapeutic, vaccine, adoptive T-cell therapeutic, or oncolytic virus to the subject. In some embodiments, the subject has a solid tumor or hematological cancer. In some embodiments, the solid tumor or hematological cancer is melanoma, breast cancer, lung cancer, colorectal cancer, liver cancer, gastric cancer, esophageal cancer, pancreatic cancer, head and neck cancer, ovary cancer, cervical cancer, urothelial cancer, renal cell cancer, bladder cancer, prostate cancer, lymphoma, and/or leukemia. In some embodiments, the subject has a viral infection or bacterial infection.

In some embodiments, the method further comprises administering immunotherapy to the subject. In some embodiments, the immunotherapy comprises administering an immune checkpoint blockade binding agent to the subject. In some embodiments, the immune checkpoint blockade binding agent is antibody comprising one or more of an anti-CTLA4 antibody, an anti-PD1 antibody, an anti-PD-L1 antibody, an anti-LAG-3 antibody, an anti-TIM-3 antibody, an anti-TIGIT antibody, an anti-CD47 antibody, or an anti-VISTA antibody. In some embodiments, the antibody is a human antibody, chimeric antibody, humanized antibody, an F(ab)′2, an Fab, an Fv, a single domain antibody, a bispecific antibody, a helix-stabilized antibody, a single-chain antibody molecule, a disulfide stabilized antibody, or a domain antibody. In some embodiments, the immunotherapy comprises administering a CAR-T cell, a CAR-NK cell, a CAR-Macrophage, a tumor vaccine, an oncolytic virus vaccine, a vaccine against an infectious agent, co-stimulatory mAb, epigenetic modulator, TLR3/7/8/9 agonist, anti-CD47, and/or IL-2 receptor agonist to the subject. In some embodiments, the DC-EVs are present within a pharmaceutical formulation.

In another aspect, the present disclosure provides a method of producing dendritic cells (DC) for use in treating a subject with cancer or with an infection, the method comprising: (a) providing a plurality of dendritic cells: (b) treating the plurality of dendritic cells so as to inhibit src homologous region 2 domain-containing phosphatase-1 (SHP1) in the cells; and (c) culturing the dendritic cells in the presence of an antigen from a tumor or an infectious agent, wherein the dendritic cells are cultured for a duration sufficient to allow the presentation of the antigen or a fragment thereof on the surface of the dendritic cells, and wherein the dendritic cells are cultured under conditions permissible for exosome production.

In some embodiments, the method further comprises: (d) harvesting extracellular vesicles from the dendritic cells; and (e) concentrating the collected dendritic cell-derived extracellular vesicles (DC-EVs). In some embodiments, the dendritic cells are monocyte-derived dendritic cells (MoDCs) or induced pluripotent stem cell (iPSC)-derived dendritic cells (iPSC-DCs). In some embodiments, the plurality of dendritic cells are treated by contacting the cells with a small molecule SHP1 inhibitor, by silencing SHP1 in the cells, or by editing the SHP1 gene in the cells so as to reduce or eliminate its expression. In some embodiments, the small molecule SHP1 inhibitor is vitamin E (DL-α-tocopherol) or an analog or variant thereof.

In some embodiments, the small molecule SHP1 inhibitor comprises the structure

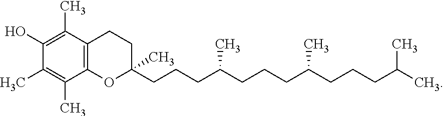 [SMILES: Cc1c(C)c2c(c(C)c1O)CC[C@@](C)(CCC[C@H](C)CCC[C@H](C)CCCC(C)C)O2]

In some embodiments, the small molecule SHP1 inhibitor is TPI-1, NSC-87877, suramin, or sodium stibogluconate (SSG). In some embodiments, the SHP1 gene is edited in the cells using a CRISPR-Cas system, comprising a guide RNA that targets the SHP1 locus. In some embodiments, the antigen is: (a) a tumor antigen that is expressed on the surface of a tumor cell of the subject's cancer or an antigenically active fragment thereof, or a peptide fragment of the tumor antigen that is capable of being presented by dendritic cells to T cells; or (b) an antigen that is expressed on the surface of the infectious agent causing the infection or an antigenically active fragment thereof, or a peptide fragment of the viral or bacterial antigen that is capable of being presented by dendritic cells to T cells.

In another aspect, the present disclosure provides a dendritic cell produced using any of the methods provided herein.

In another aspect, the present disclosure provides extracellular vesicles harvested from any of the dendritic cells provided herein.

In another aspect, the present disclosure provides a pharmaceutical formulation comprising any of the dendritic cells or extracellular vesicles provided herein.

A better understanding of the nature and advantages of embodiments of the present disclosure may be gained with reference to the following detailed description and the accompanying drawings.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1G show that dietary VitE augments ICT efficacy dependent on DCs according to various aspects of this disclosure. FIG. 1A: Percentages of deceased cancer patients taking dietary nutritional supplements during ICT treatment (Anti-PD-1/PDL1: Pembrolizumab/Atezolizumab) (n=7,667 including 1,888 breast cancer patients, 2,715 melanoma patients, and 3,064 prostate cancer patients). Each bar represents a group of patients who took one type of nutritional supplement along with ICT (Red dashed line indicated the median percentage of deceased cancer patients in this study). FIG. 1B: Percentages of deceased cancer patients who took VitE (8.4%) (n=83) during anti-PD-1/PD-L1 treatment compared with cancer patients who did not (21.2%) (n=7, 574) was shown (Fisher exact test P-value 0.0027). FIGS. 1C-1D: Tumor growth curves of orthotopic EMT6 TNBC tumor-bearing Balb/c mice (FIG. 1C, n=5) and orthotopic B16-GMCSF melanoma tumor-bearing C57BL/6 mice (FIG. 1D, left, n=5) treated with vehicle, 50 mg/kg of Vitamin E (VitE), 200 mg/mouse anti-PDI (ICT), or Vitamin E+anti-PD1 (VitE+ICT) (n=5). Flow cytometry quantification of Granzyme B⁺CD8⁺ T cells in tumor tissues at 20 days post-implantation (FIG. 1D. right, n=5). FIG. 1E: Tumor growth curves of orthotopic B16-F10 melanoma tumor-bearing C57BL/6 mice with the indicated treatments (n=5). FIG. 1F: Tumor growth curves of orthotopic B16-FLT3L melanoma tumor-bearing C57BL/6 mice with the indicated treatments (n=5). FIG. 1G: Tumor growth curves (left) and Kaplan-Meier curves (right) of C57BL/6 mice bearing B16-GMCSF tumors treated with vehicle (n=4), ICT+VitE (n=8), or ICT+VitE plus Cyt c that depletes DCs (n=10). Experiments were repeated at least twice to observe concordant statistical significance defined by P-value: ns. no significant. *P<0.05, **P<0.01, ***P<0.001, ****P<0.0001.

FIGS. 2A-2C show that VitE enhances DCs activation in vitro and reinvigorated DCs function in vivo according to various aspects of this disclosure. FIG. 2A: Percentage of human IFN-γ⁺CD8⁺ T cells in CD8⁺ T cells in coculture with vehicle or VitE-treated autologous MoDCs (n=3). FIG. 2B: Representative flow cytometric analysis and quantification of antigen-specific OT-1 T cell proliferation stimulated with OVA-loaded mouse BMDCs that were treated with vehicle or VitE at day 3. FIG. 2C: Tumor growth curves of orthotopic EMT6 tumor-bearing mice treated with vehicle (Ctrl), Vitamin E (VitE), or VitE plus Cyt c (10 mg/kg. i.v, every other day starting on day 3) to deplete DCs (n=8/group). The statistical significance is defined by P-value: *P<0.05, **P<0.01, ***P<0.001. ****P<0.0001.

FIGS. 3A-3F show that VitE restores TADCs function by inhibiting SHP1 activity through SCARB1 according to various aspects of this disclosure. FIG. 3A: Volcano plot of reverse phase protein array (RPPA) data of mouse BMDCs treated with VitE (50 μg/ml) compared to those treated with vehicle control with tumor conditioned media (TCM) for 48 hours (n=3 independent samples). Differentially regulated proteins are labeled, and the top hits are marked in red (up-regulated proteins) or blue (down-regulated proteins). The grey dashed line indicates P-value of 0.05. FIG. 3B: t-SNEs by CyTOF and quantification comparing the intensity of pY564-SHP1 in TADCs from the vehicle-or VitE-treated EMT6 tumors at day 15 post-implantation. FIG. 3C: Top, SHP1 knockdown in BMDCs was effective. Bottom, CFSE-dilution analysis of OT-1 T cells co-cultured with OVA protein-pulsed BMDCs (ratio 10:1) with knockdown of SHP1 by siPtpn6 versus siCtrl (n=3). Right, quantification of OT-1 T-cell numbers under the indicated condition. FIG. 3D: The binding of VitE to purified SHP1 is analyzed by microscale thermophoresis (MST) binding assay. The apparent dissociation constant Kd is 39±26 μM (mean of triplicates). FIG. 3E: Relative tyrosine phosphatase activity of SHP1 immunoprecipitated from LPS (100 ng/ml)-activated DC2.4 cells in the presence of indicated doses of VitE (mean of triplicates). FIG. 3F: SHP1 tyrosine phosphatase activity in LPS (100 ng/ml)-activated BMDCs (n=6) treated with vehicle-or VitE (50 μg/ml). Error bars, s.e.m. ns, no significant. *P<0.05, **P<0.01. ***P<0.001, ****P<0.0001.

FIGS. 4A-4B show that SHP1 inhibition enhances the antigen cross-presentation of DCs according to various aspects of this disclosure. FIG. 4A: Flow cytometric analysis and quantification of H-2Kb-SIINFEKL in OVA-loaded sgCtrl or sgPtpn6-DC2.4 cells without or with TCM treatment (n=3). FIG. 4B: FCM analysis and quantification of H-2K^{b}-SIINFEKL in the vehicle or VitE-treated BMDCs with or without TCM treatment (n=5). Error bars, s.e.m. ns, no significant. *P<0.05, **P<0.01. ***P<0.001. ****P<0.0001.

FIGS. 5A-5G show that SHP1 inhibition enhances the antigen cross-presentation of DC-derived EVs and boosts system antitumor immunity according to various aspects of this disclosure. FIG. 5A: The representative transmission electron microscope (TEM) image of DC-EVs (top) and flow cytometric analysis of H-2K^{b}-SIINFEKL in DC-EVs (bottom) derived from OVA-loaded BMDCs treated with vehicle or VitE. FIG. 5B: Quantification (n=5) of DiO³⁰ CD8⁺ T cells after incubating T-cells with vehicle-or VitE-treated DiO-labelled DC-EVs. FIG. 5C: Flow cytometry analysis of the proliferation of OT-1 T cells cultured with the addition of EVs derived from OVA-loaded vehicle-DC-EVs or VitE-DC-EVs (left panel). IFNγ production by OT-1 T cells with the indicated treatment was measured by ELISA (right panel, n=5). FIG. 5D: Tumor volume and survival of orthotopic E0771-OVA tumor-bearing mice treated with PBS (Vehicle), vehicle-DC-EVs. or VitE-DC-EVs (30 μg/mouse by i.v. at days 2, 9, 16.) (n=6). FIG. 5E: Tumor volume and survival of E0771-OVA tumor-bearing mice treated with ICT (100 μg/mouse anti-CTLA4 and 200 μg/mouse anti-PDI by i.p. at day 7, 10, 13) plus PBS, sgCtrl-DC2.4-EVs, or sgPtpn6-DC2.4-EVs (30 μg/mouse by i.v. at day 2, 9, 16) (n=8). FIGS. 5F-G: Tumor growth curves of subcutaneous B16-GMCSF-OVA tumor-bearing mice treated with vehicle, sgCtrl-or sgPtpn6-DC2.4-EVs (30 μg/mouse by i.v. at day 2, 9, 16) (FIG. 5F, n =6) and treated with ICT (as FIG. 5E above) in combination with the vehicle, sgCtrl-DC2.4-EVs or sgPtpn6-DC2.4-EVs (FIG. 5G, n=6). Error bars, s.e.m. ns, no significant. *P<0.05, **P<0.01, ***P<0.001. ****P<0.0001.

FIGS. 6A-6F show that VitE enhances immune induction therapies to boost immunotherapy according to various aspects of this disclosure. FIG. 6A: Diagram of the experimental paradigm (top) and tumor growth curves (bottom) of orthotopic B16-GMCSF tumor in mice vaccinated with or without GVAX (s.c. at day 1, 4, 7), then treated with vehicle or VitE (50 mg/kg/day daily by oral gavage administration from day 4 to day 20) (n=6/group). FIG. 6B: Tumor weight of 4T1 tumors from mice treated with vehicle. GVAX, ICT (200 μg/mouse anti-PDI and 100 μg/mouse anti-CTLA4 by i.p. at day 7, 10, 13), or GVAX+ICT in combination with vehicle or VitE (50 mg/kg/day daily by oral gavage administration from day 4 to day 25) (n=4) at day 25 after tumor inoculation. FIG. 6C: Flow cytometric analysis (left) and quantification (right) of gp70 tetramer+CD8⁺ T cells in 4T1 tumors at day 14 post-implantation with indicated treatments (n=5/group). FIG. 6D: Kaplan-Meier survival curve of 4T1 tumor-bearing mice treated with vehicle, low dosage of Doxorubicin (Dox, 5 mg/kg by i.v.). ICT (200 μg/mouse anti-PD1 and 100 μg/mouse anti-CTLA4 by i.p. at day 7, 10, 13). or Dox+ICT along with vehicle or VitE (n=10). FIG. 6E: Measurement of maximal tumor diameter in HY1936 KPC tumor-bearing mice treated with gemcitabine (GEM, 25 mg/kg by i.p.) and ICT (anti-PDI/anti-CTLA4) along with vehicle or VitE (n=5). FIG. 6F: Kaplan-Meier survival curve of HY19636 KPC tumor-bearing mice treated with vehicle, GEM (gemcitabine, 25 mg/kg by i.p.), GEM+ICT (anti-PDI/anti-CTLA4) plus vehicle or VitE (n=6). Error bars, s.e.m. ns, no significant. *P<0.05. ***P<0.001, ****P<0.0001.

FIGS. 7A-7J show that DC-EVs from VitE-treated or SHP1 -reduced DCs boost anti-tumor T cell immunity in vitro and in vivo according to various aspects of this disclosure. FIG. 7A: OT-1 T cell proliferation cultured with the addition of EVs derived from sgCtrl-or sgPtpn6-DC2.4 cells in the absence or presence of TCM (n=4). FIG. 7B: Diagram of the experimental paradigm (FIG. 7B) of EO771-OVA tumor-bearing mice treated with PBS (i.v., Vehicle), vehicle-DC-EVs or VitE-DC-EVs (30 μg/mouse by i.v. at day 2, 9, 16). FIG. 7C. Ratio of CD8⁺/Treg (CD4′FoxP3+) in CD45+TILs and OVA-tetramer CD8⁺ T cell populations in EO771-OVA tumor-bearing mice treated with PBS (Vehicle), vehicle-DC-EVs, or VitE-DC-EVs (n=5-6) at day 30, detected by flow cytometry. FIG. 7D: Mean tumor volume of E0771-OVA tumor-bearing mice treated with vehicle. sgCtrl-DC2.4-EVs, or sgPtpn6-DC2.4-EVs (n=6). FIG. 7E and FIG. 7F: Diagram of the experimental paradigm (FIG. 7E) and lung metastasis (FIG. 7F) of E0771-OVA tumor-bearing mice treated with vehicle, sgCtrl-DC2.4-EVs or sgPtpn6-DC2.4-EVs in combination with ICT (n=8). FIG. 7G: Tumor image and volume of subcutaneous B16-GMCSF-OVA tumor-bearing mice treated with vehicle, sgCtrl--DC2.4-EVs or sgPtpn6-DC2.4-EVs (n=5-6). FIG. 7H and FIG. 7I: Diagram of the experimental paradigm (FIG. 7H), tumor images (FIG. 71, n=5) of B16-GMCSF-OVA tumor-bearing mice treated with vehicle, ICT in combination with the vehicle. sgCtrl-DC2.4-EVs or sgPtpn6-DC2.4-EVs at day 22 post-implantation. FIG. 7J: Flow cytometry quantification of OVA-tetramer'CD8⁺ T cell populations in 4 groups of B16-GMCSF-OVA tumors in (FIG. 7I). Error bars, s.e.m. ns, no significant. *P<0.05.**P<0.01. ***P<0.001.

FIGS. 8A-8I show that VitE further enhances immune induction therapies to increase responses to immunotherapy according to various aspects of this d isclosure. FIG. 8A: Flow cytometric quantification of GZMB+CD8⁺ T cell populations in B16-GMCSF tumors at day 14 post-implantation with indicated treatments (n=3). FIG. 8B and FIG. 8C: Diagram of the experimental paradigm (FIG. 8B) and quantification of CD44³⁰ CD62L⁻CD8⁺ T cells subpopulations (FIG. 8C) in orthotopic 4T1 tumors from mice treated with vehicle, GVAX (by subcutaneous injection at day 1, 4, 7), ICT (200 μg/mouse anti-PD1 and 100 μg/mouse anti-CTLA4 at day 7, 10, 13), or GVAX+ICT in combination with vehicle or VitE (50 mg/kg/day daily by oral gavage administration) (n=3-5). FIG. 8D to 8F: Individual tumor growth curves (FIG. 8D). lung metastasis (FIG. 8E), and flow cytometry quantification of gp70 tetramer⁺CD8⁺ T cell populations (FIG. 8F) of 4T1 tumor-bearing mice treated with the indicated conditions (n=5-10). FIG. 8G to 8I: Diagram of the experimental paradigm (FIG. 8G), tumor weight (FIG. 8H), and ascites volume (FIG. 8I) of HY 19636 KPC tumor-bearing mice with indicated treatment at day 25 post-implantation (n=5). Error bars, s.e.m. ns, no significant. *P<0.05. **P<0.01, ***P<0.001.

FIGS. 9A-9B show that the inhibition of SHP1 in dendritic cells (DCs) increases antitumor immunity and delays tumor growth in breast cancer brain metastasis (BrM) according to various aspects of this disclosure. FIG. 9A: Representative brain metastasis (BrM) lesions of C57BL/6 mice after the internal carotid artery (ICA)-injected with E0771-OVA-GFP-Luc tumor cells alone, or co-implanted with sgCtrl-or sgPtpn6-DC2.4 cells at day 20 post-implantation. FIG. 9B: Flow cytometry analysis of OVA-tetramer'CD8⁺ T cell populations in BrM lesions or spleen of C57BL/6 mice after ICA-injected with indicated E0771-OVA-GFP-Luc tumors alone or with sgCtrl-or sgPtpn6-DC2.4 cells at day 20 post-implantation.

FIGS. 10A-10C show that the pharmacological inhibition of SHP1 by vitamin E or TPI-1 enhances the activation of dendritic cells (DCs) derived from human induced pluripotent stem cells (iPSCs) according to various aspects of this disclosure. FIG. 10A: Representative high magnification photomicrographs (100×, 200×, 400×) illustrating the morphology of colonies and individual cells of fully differentiated iPSC-derived DCs (iPSC-DCs) displaying characteristic DC morphology consisting of protrusions and veils of cytoplasm at the end of the differentiation procedure (day 23) after using the differentiation protocol for the addition and withdrawal of growth factors and cytokines. FIG. 10B: Phenotypic characterization of generated human iPSC-DCs. Representative flow cytometry analysis depicting the expression of DC lineage markers by iPSC-DCs gated on the CD11c+ population. Levels of expression of individual markers are shown as red histograms, while non-specific staining by isotype controls is shown as light blue histograms. FIG. 10C: Phenotypic characterization of human iPSC-DCs after treatment with vitamin E or TPI-1 to inhibit SHP1. Representative flow cytometry analysis (left panel) and quantification (right panel) depicting the expression of indicated activity markers by iPSC-DCs gated on the CD11c+population with the treatment of vehicle, vitamin E (50 μg/ml) or TPI-1 (1 μg/ml) for 72 h.

### DETAILED DESCRIPTION

### A. Introduction

The present disclosure provides methods and compositions for the treatment of cancer or infection in a subject by administering dendritic cell (DC)-derived extracellular vesicles (DC-EVs) obtained from DCs that have been treated to inhibit src homologous region 2 domain-containing phosphatase-1 (SHP1) and that have been loaded with an antigen present in the cancer of the subject or in an infectious agent present in the subject. DC-EVs produced using the herein-described methods have the ability to transfer preformed functional MHC-peptide complexes from DCs to recipient T cells, which facilitate T cell-dependent tumor rejection and the targeting of infectious disease agents. DC-EVs present distinct advantages over cell-based immunotherapies involving DCs, e.g., concerning safety, targeting capabilities, and clinical practicality.

In some embodiments, the provided methods and compositions improve the efficacy of immune modulation therapies for cancer, infections, and other immune-related diseases and conditions. In particular, the present methods and compositions help overcome the limitations of DC-EV-based immune modulation through the inhibition of SHP1 in dendritic cells. SHP1, a cellular tyrosine phosphatase encoded by the protein tyrosine phosphatase, non-receptor type 6 (Ptpn6) gene, inhibits phosphorylation of Syk and its downstream MAPKs, e.g., p-ERKI/2 and p-c-JUN (Abram et al., 2013; Carmi et al., 2016), and is expressed by all mature hematopoietic lineages and at low levels, in a different isoform, by endothelial cells (Lorenz, 2009).

The present disclosure is based upon the surprising discovery that SHP1 is a pleiotropic inhibitor of DC function and its inhibition in DCs enhances the strength of immune responses. Without being bound by the following theory, it is believed that a major reason why previous clinical trials with DC-EVs have shown poor results is because of a dysfunction in the DCs, leading to a lack of adaptive immune response, particularly anti-tumor CD8⁺ T cell response. In particular, it is believed that DCs are profoundly dysfunctional upon exposure to the suppressive tumor microenvironment (TME), as indicated by low levels of cancer-specific peptide-MHC-I (pMHC-I) complexes in DCs and DC-EVs. and this can limit the efficacy of T-cell-dependent ICT. Accordingly, overcoming DC-suppressive signals using the present methods can potentiate the T-cell antitumor response and enhance, e.g., the effects of ICT.

For example, during in vitro generation, monocyte-derived DCs or tumor-associated dendritic cells (TADCs) are mostly dysfunctional due to upregulated pSHP1/SHP1, limiting its antigen presentation and antitumor T-cell priming and activation function. The present disclosure provides methods and compositions for modifying DCs with SHP1 inhibitors such as vitamin E (VitE) or genetic SHP1-knockdown/knockout, thereby promoting the immunogenic capacity of DCs and DC-EVs. In addition, SHP1-inhibited DC-EVs can be used to enhance immune responses in the treatment of other diseases, e.g., pathogen (virus, bacteria) infectious diseases (Kowal and Tkach, 2019).

Also disclosed herein are methods of producing SHP-modified DCs and DC-EVs, as well as DCs and DC-EVs produced using the methods. Also provided are methods of using these DCs and extracellular vesicles to treat cancer, infections, and other immune system-related diseases.

### B. Preparation of Dendritic Cells

### 1. Isolation of Cells

The present methods involve the use of dendritic cells (DCs) that have been treated so as to inhibit SHP1 in the cells, and/or extracellular vesicles isolated from the cells. The dendritic cells can be isolated from any of a number of sources. In particular embodiments, the dendritic cells are monocyte-derived dendritic cells (MoDCs) or are derived from induced pluripotent stem cells (iPSCs; the dendritic cells derived from the iPSCs are referred to as ipDCs). MoDCs can be generated, e.g., by applying peripheral blood of healthy individuals to a Ficoll gradient (BD), isolating mononuclear cells, and culturing them in a humidified incubator for 1 hour to let monocytes adhere. After removing the medium with non-adherent cells, the adherent monocytes are collected and then differentiated into MoDCs, e.g., by culturing the monocytes in appropriate DC culture medium (e.g., RPMI 1640 containing L-glutamine, HEPES, sodium Pyruvate, MEM nonessential amino acids, penicillin/streptomycin, 2-mercaptoethanol, and FBS, supplemented with GM-CSF and IL-4). The media is refreshed, e.g., with supplement cytokines every 3 days. Once the majority of monocytes have differentiated into immature DCs, they can be re-cultured in fresh DC culture medium supplemented with tumor necrosis factor-alpha (TNF-α) for an additional day to generate mature DCs.

DCs can also be derived from bone marrow. In one embodiment, bone marrow (BM) is harvested from a mammal (e.g., mice) by flushing femurs, tibias, and/or humeri with DC culture medium. Bone-marrow cells are strained through a filter, centrifuged, and resuspended in RBC lysis buffer. The remaining cells are plated in DC culture medium supplemented with, e.g., mGM-CSF and mIL-4, and the media refreshed with supplement cytokines every 3 days. Once the majority of BM cells (>90%) are differentiated into DCs, the BMDCs are re-cultured in fresh DCs culture medium supplemented with lipopolysaccharide for an additional day to generate mature DCs.

### 2. Inhibition of SHP1

Once DCs have been isolated, they are treated to inhibit SHP1 in the cells. The protein sequence for human SHP1 (also referred to as Tyrosine-protein phosphatase non-receptor type 6, or PTPN6) is shown as UniProt ID P29350, and the nucleic acid sequence is shown as NCBI Gene ID 5777, the entire disclosures of which are herein incorporated by reference. Any agent that reduces, decreases, counteracts, attenuates, inhibits, blocks, downregulates, or eliminates in any way the expression, stability or activity (e.g., phosphatase activity) of SHP1 in dendritic cells can be used in the present methods. Inhibitors can be small molecule compounds, peptides, polypeptides, nucleic acids, antibodies, e.g., blocking antibodies or antibody fragments, or any other molecule that reduces, decreases, counteracts, attenuates, inhibits, blocks, downregulates, or eliminates in any way the expression, stability and/or activity of SHP1. In particular embodiments, SHP1 is inhibited using a small molecule inhibitor such as Vitamin E, using gene silencing, or by modifying the SHP1 gene using a CRISPR-Cas system so as to reduce or eliminate its expression.

In some embodiments, the SHP1 inhibitor decreases the activity (e.g., phosphatase activity), stability or expression of SHP1 by at least 10%, 15%, 20%, 25%, 30%, 35%, 40%, 45%, 50%, 55%, 60%, 65%, 70%, 75%, 80%, 85%, 90%, or more relative to a control level, e.g., a level determined in the absence of the inhibitor, in vivo or in vitro.

The efficacy of inhibitors can be assessed in any of a variety of ways, including in vitro and in vivo methods. For example, the phosphatase activity of SHP1 can be assessed using an assay such as provided by the RediPlate 96 EnzChek Tyrosine Phosphatase Assay Kit (ThermoFisher). Inhibition can also be measured indirectly, e.g., by measuring phosphorylation at tyrosine 564 (Y564) of SHP1, or by detecting the activity and/or phosphorylation of downstream signaling molecules such as Syk and its downstream MAPKs, e.g., p-ERK1/2 and p-c-JUN (Abram et al., 2013: Carmi et al., 2016).

In some embodiments, the SHP1 inhibitor is considered effective if the phosphatase activity as described herein is decreased by at least 10%, at least 20%, at least 30%, at least 40%, at least 50%, at least 60%, at least 70%, at least 80%, at least 90% or more as compared to the reference value, e.g., the value in the absence of the inhibitor, in vitro or in vivo. In some embodiments, a SHP1 inhibitor (e.g., an RNAi molecule) is considered effective if the level of expression of a SHP1-encoding polynucleotide is decreased by at least 1.5-fold, at least 2-fold, at least 3-fold, at least 4-fold, at least 5-fold, at least 6-fold, at least 7-fold, at least 8-fold, at least 9-fold, at least 10-fold or more as compared to the reference value.

In some embodiments, the SHP1 inhibitor is considered effective if it improves one or more immune features of dendritic cells, e.g., the ability to activate T cells (e.g., CD8⁺ T cells) following loading of the dendritic cells with an antigen as described herein. In some such embodiments, the inhibitor results in an increase in dendritic cell activation of T cells of, e.g., 5%, 10%, 20%, 30%, 40%, 50%, 60%, 70%, 80%, 90%, or more relative to the level in dendritic cells prior to the administering of the SHP 1 inhibitor.

In some embodiments, the inhibitor results in an increase or decrease in the activity and/or phosphorylation of a molecule downstream of SHP1, such as Syk, p-ERK1/2, or p-c-JUN relative to the level in the dendritic cells prior to the administering of the SHP1inhibitor, e.g., an increase or decrease of 5%, 10%, 20%, 30%, 40%, 50%, 60%, 70%, 80%, 90%, or more relative to the level in the cells prior to the administering of the SHP1 inhibitor.

The efficacy of inhibitors can also be assessed, e.g., by detection of decreased polynucleotide (e.g., SHP1 mRNA) expression, which can be analyzed using routine techniques such as RT-PCR. Real-Time RT-PCR, semi-quantitative RT-PCR, quantitative polymerase chain reaction (qPCR), quantitative RT-PCR (qRT-PCR), multiplexed branched DNA (bDNA) assay, microarray hybridization, or sequence analysis (e.g., RNA sequencing (“RNA-Seq”)). Methods of quantifying polynucleotide expression are described, e.g., in Fassbinder-Orth, Integrative and Comparative Biology, 2014, 54:396-406; Thellin et al., Biotechnology Advances, 2009, 27:323-333; and Zheng et al., Clinical Chemistry, 2006, 52:7 (doi: 10/1373/clinchem.2005.065078). In some embodiments, real-time or quantitative PCR or RT-PCR is used to measure the level of a polynucleotide (e.g., mRNA) in a biological sample. See, e.g., Nolan et al., Nat. Protoc, 2006, 1:1559-1582: Wong et al., BioTechniques, 2005, 39:75-75. Quantitative PCR and RT-PCR assays for measuring gene expression are also commercially available (e.g., TaqMan® Gene Expression Assays, ThermoFisher Scientific).

In some embodiments, the SHP1 inhibitor is considered effective if the level of expression of a SHP1-encoding polynucleotide is decreased by at least 10%, at least 20%, at least 30%, at least 40%, at least 50%. at least 60%. at least 70%, at least 80%, at least 90% or more as compared to the reference value, e.g., the value in the absence of the inhibitor, in vitro or in vivo. In some embodiments, a SHP1 inhibitor is considered effective if the level of expression of a SHP1 -encoding polynucleotide is decreased by at least 1.5-fold, at least 2-fold, at least 3-fold, at least 4-fold, at least 5-fold, at least 6-fold, at least 7-fold, at least 8-fold, at least 9-fold, at least 10-fold or more as compared to the reference value.

The effectiveness of a SHP1 inhibitor can also be assessed by detecting protein expression or stability, e.g., using routine techniques such as immunoassays, two-dimensional gel electrophoresis, western blot, and quantitative mass spectrometry, all of which are known to those skilled in the art. Protein quantification techniques are generally described in “Strategies for Protein Quantitation,” Principles of Proteomics, 2nd Edition, R. Twyman, ed., Garland Science, 2013. In some embodiments, protein expression or stability is detected by immunoassay, such as but not limited to enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); immunofluorescence (IF); fluorescence polarization immunoassay's (FPIA); and chemiluminescence assays (CL). If desired, such immunoassay's can be automated. Immunoassays can also be used in conjunction with laser induced fluorescence (see, e.g., Schmalzing et al., Electrophoresis, 18:2184-93 (1997): Bao, J. Chromatogr. B. Biomed. Sci., 699:463-80 (1997)).

For determining whether a SHP1 protein levels are decreased in the presence of a SHP1 inhibitor, the method comprises comparing the level of the protein (e.g., SHP1 protein) in the presence of the inhibitor to a reference value, e.g., the level in the absence of the inhibitor. In some embodiments, a SHP1 protein is decreased in the presence of an inhibitor if the level of the SHP1 protein is decreased by at least 10%, at least 20%, at least 30%, at least 40%, at least 50%, at least 60%, at least 70%, at least 80%, at least 90% or more as compared to the reference value. In some embodiments, a SHP1 protein is decreased in the presence of an inhibitor if the level of the SHP1 protein is decreased by at least 1.5-fold, at least 2-fold, at least 3-fold, at least 4-fold, at least 5-fold, at least 6-fold, at least 7-fold, at least 8-fold, at least 9-fold, at least 10-fold or more as compared to the reference value.

### a) Small Molecules

In some embodiments of the invention, SHP1 is inhibited by the administration of a small molecule inhibitor. Any small molecule inhibitor can be used that reduces, e.g., by 10%, 15%, 20%, 25%, 30%, 35%, 40%, 45%, 50%, 55%, 60%, 65%, 70%, 75%, 80%, 85%, 90%, or more, the expression, stability or activity of SHP1 relative to a control, e.g., the expression, stability or activity in the absence of the inhibitor. In particular embodiments, small molecule inhibitors are used that can bind to SHP1, e.g., in the central cavity formed at the interface of all three domains of SHP1 (N-SH2, C-SH2, and PTP domains).

In particular embodiments, the small molecule is Vitamin E (D-α-Tocopherol type VI, Cat. T1539, Sigma-Aldrich), or a derivative, variant, or analog thereof (e.g., any of alpha-, beta-, gamma-, and delta-tocopherols or alpha-, beta-, gamma-, and delta-tocotrienols, or variants thereof). In some embodiments, the small molecule is the specific SHP1 inhibitor TPI-1 (Tyrosine phosphatase inhibitor 1:2-(2,5-dichlorophenyl)-2,5-cyclohexadiene-1,4-dione) (Cat. 22480, Cayman). In some embodiments, the small molecule is any of the inhibitors disclosed in Kundu et al. (2010) J. Immunol. 184(11): 6529-36, the entire disclosure of which is herein incorporated by reference. Other suitable inhibitors include non-specific SHP1 inhibitors such as NSC-87877 (8-Hydroxy-7-[(6-sulfo-2-naphthyl) azo]-5-quinolinesulfonic acid) (Cat. 2613, Tocris; Cat. 14908, Cayman), suramin (8-[[4-methyl-3-[[3-[[3-[[2-methyl-5- [(4,6,8-trisulfonaphthalen-1-yl) carbamoyl]phenyl]carbamoyl]phenyl]carbamoylamino]benzoyl]amino]benzoyl]amino]naphthalene-1,3,5-trisulfonic acid) (Cat. BD63032, BLD Pharm; Cat. DC24155, DC Chemicals), and sodium stibogluconate (SSG) (Cat. MC528439, Molcore; DY528439, 001 Chemical).

In some embodiments, the molecule comprises the structure:

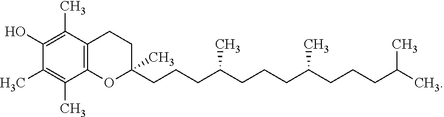 [SMILES: Cc1c(C)c2c(c(C)c1O)CC[C@@](C)(CCC[C@H](C)CCC[C@H](C)CCCC(C)C)O2]

Candidate small molecule (and other) inhibitors can be identified and/or assessed using molecular docking analysis. For example, high-resolution crystal structures of auto-inhibitory SHP1 (PDB ID: 2B3O) can be obtained from Protein Data Bank. The 3D structures of candidate SHP1 inhibitors, such as VitE (αT), can be obtained, e.g., from PubChem (e.g., for VitE, PubChem CID: 14985). The SHP1 structure and candidate inhibitor's structure can be uploaded to, e.g., Webina—an AutoDock-based webserver (durrantlab.pitt.edu/webina/), and the interaction of the inhibitor and SHP1 can be assessed for various possible models. For example, for all the top 9 docking complex models, VitE is bound in the central cavity of SHP1, formed at the interface of all three domains of SHP1 (N-SH2,C-SH2, and PTP domains).

### b) Inhibitory Nucleic Acids

In some embodiments, the SHP1 inhibitor comprises an inhibitory nucleic acid, e.g., antisense DNA or RNA, small interfering RNA (siRNA), microRNA (miRNA), or short hairpin RNA (shRNA). In some embodiments, the inhibitory RNA targets a sequence that is identical or substantially identical (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical) to a target sequence in a SHP1 polynucleotide (e.g., a portion comprising at least 20, at least 30, at least 40, at least 50, at least 60, at least 70, at least 80, at least 90, or at least 100 contiguous nucleotides, e.g., from 20-500, 20-250, 20-100, 50-500, or 50-250 contiguous nucleotides of a SHP1 gene (e.g., the human PTPN6 gene, NCBI Gene ID: 5777).

In some embodiments, the methods described herein comprise silencing the PTPN6 gene using an shRNA or siRNA. A shRNA is an artificial RNA molecule with a hairpin turn that can be used to silence target gene expression via the siRNA it produces in cells. See, e.g., Fire et. al., Nature 391:806-811, 1998; Elbashir et al., Nature 411:494-498, 2001; Chakraborty et al., Mol Ther Nucleic Acids 8:132-143, 2017; and Bouard et al., Br. J. Pharmacol. 157:153-165, 2009. In some embodiments, a dendritic cell is contacted with a modified RNA or a vector comprising a polynucleotide that encodes an shRNA or siRNA capable of hybridizing to a portion of an SHP1 mRNA. In some embodiments, the vector further comprises appropriate expression control elements known in the art, including, e.g., promoters (e.g., inducible promoters or tissue specific promoters), enhancers, and transcription terminators.

In some embodiments, the inhibitor is a SHP1 -specific microRNA (miRNA or miR). A microRNA is a small non-coding RNA molecule that functions in RNA silencing and post-transcriptional regulation of gene expression. miRNAs base pair with complementary sequences within the mRNA transcript. As a result, the mRNA transcript may be silenced by one or more of the mechanisms such as cleavage of the mRNA strand, destabilization of the mRNA through shortening of its poly(A) tail, and decrease in the translation efficiency of the mRNA transcript into proteins by ribosomes.

In some embodiments, the inhibitor is an antisense oligonucleotide, e.g., an RNase H-dependent antisense oligonucleotide (ASO). ASOs are single-stranded, chemically modified oligonucleotides that bind to complementary sequences in target mRNAs and reduce gene expression both by RNase H-mediated cleavage of the target RNA and by inhibition of translation by steric blockade of ribosomes. In some embodiments, the oligonucleotide is capable of hybridizing to a portion of a SHP1 mRNA. In some embodiments, the oligonucleotide has a length of about 10-30 nucleotides (e.g., 10, 12, 14, 16, 18, 20, 22, 24, 26, 28, or 30 nucleotides). In some embodiments, the oligonucleotide has 100% complementarity to the portion of the mRNA transcript it binds. In other embodiments, the DNA oligonucleotide has less than 100% complementarity (e.g., 95%, 90%, 85%, 80%, 75%, or 70% complementarity) to the portion of the mRNA transcript it binds, but can still form a stable RNA: DNA duplex for the RNase H to cleave the mRNA transcript.

Suitable antisense molecules, siRNA, miRNA, and shRNA can be produced by standard methods of oligonucleotide synthesis or by ordering such molecules from a contract research organization or supplier by providing the polynucleotide sequence being targeted. The manufacture and deployment of such antisense molecules in general terms may be accomplished using standard techniques described in contemporary reference texts: for example, Gene and Cell Therapy: Therapeutic Mechanisms and Strategies, 4^{th} edition by N. S. Templeton: Translating Gene Therapy to the Clinic: Techniques and Approaches, 1^{st} edition by J. Laurence and M. Franklin; High-Throughput RNAi Screening: Methods and Protocols (Methods in Molecular Biology) by D.O. Azorsa and S. Arora: and Oligonucleotide-Based Drugs and Therapeutics: Preclinical and Clinical Considerations by N. Ferrari and R. Segui.

Inhibitory nucleic acids can also include RNA aptamers, which are short, synthetic oligonucleotide sequences that bind to proteins (see, e.g., Li et al., Nuc. Acids Res. (2006), 34:6416-24). They are notable for both high affinity and specificity for the targeted molecule, and have the additional advantage of being smaller than antibodies (usually less than 6 kD). RNA aptamers with a desired specificity are generally selected from a combinatorial library, and can be modified to reduce vulnerability to ribonucleases, using methods known in the art.

In some embodiments, endoribonuclease-prepared siRNAs (esiRNAs) are used to inhibit SHP1. esiRNAs are a mixture of siRNA oligos resulting from cleavage of long double-stranded RNA (dsRNA) with an endoribonuclease such as Escherichia coli RNase III or Dicer. esiRNAs are a heterogeneous mixture of siRNAs that all target the same mRNA sequence (e.g., SHP1 , or PTPN6). In one embodiments, MISSION® esiRNA is used for targeting Ptpn6 in BMDCs.

### c) Genetic Modification

In some embodiments, the PTPN6 gene (encoding SHP1) is inhibited by genomic modification, e.g., by deleting the PTPN6 gene in a DC or by introducing a mutation in the gene that, e.g., decreases or abolishes its expression, activity, or stability. Such methods can be carried out using any suitable method known in the art, e.g., using a CRISPR-Cas system. The CRISPR-Cas system comprises at least one guide RNA (typically a single guide RNA, or sgRNA), an RNA-guided nuclease (such as Cas) or Cpf1), and optionally a homologous donor template. A homologous donor template can be used to introduce specific modifications into the genome by homologous recombination. In the absence of a homologous donor template, however, cleavage of the gRNA target sequence can still inactivate a gene through the introduction of small insertions or deletions (indels).

### sgRNAS

In some embodiments, the single guide RNAs (sgRNAs) used to inhibit SHP1 is designed to target the PTPN6 locus (located, e.g., on chromosome 12 at 12p13.31 in humans). sgR.VAs interact with a site-directed nuclease such as Cas9 and specifically bind to or hybridize to a target nucleic acid within the genome of a cell, such that the sgRNA and the site-directed nuclease co-localize to the target nucleic acid in the genome of the cell. The sgRNAs as used herein comprise a targeting sequence that has homology (or complementarity) to a target DNA sequence at the PTPn6 locus, and a constant region that mediates binding to Cas9 or another RNA-guided nuclease. The sgRNA can target any sequence within PTPN6 adjacent to a PAM sequence. In some embodiments, the target sequence is within exon 3 of CCR5. In particular embodiments, the target sequence of the sgRNA comprises the sequence: Ptpn6-sgRNA1: CAAACTTCTCCCCTCCGTAC (SEQ ID NO: 1); Ptpn6-sgRNA2: GACTTCTATGACCTGTACGG (SEQ ID NO:2); or Ptpn6-sgRNA3: CTGGCCCAGGTTCCCCGCTC (SEQ ID NO:3). In some embodiments, the target sequence of the sgRNA comprises at least 80% (e.g., at least 85%, 86%, 87, %, 88%, 89%, 90%, 91%, 92%, 93%, 94%, 95%, 96%, 97%, 98%, or 99%) identity to the sequence of SEQ ID NOs: 1-3.

The targeting sequence of the sgRNAs may be, e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or, e.g., 15-25, 18-22, or 19-21 nucleotides in length, and shares homology with a targeted genomic sequence, in particular at a position adjacent to a CRISPR PAM sequence. The sgDNA targeting sequence is designed to be homologous to the target DNA, i.e., to share the same sequence with the non-bound strand of the DNA template or to be complementary to the strand of the template DNA that is bound by the sgRNA. The homology or complementarity of the targeting sequence can be perfect (i.e., sharing 100% homology or 100% complementarity to the target DNA sequence) or the targeting sequence can be substantially homologous (i.e., having less than 100% homology or complementarity, e.g., with 1-4 mismatches with the target DNA sequence).

Each sgRNA also includes a constant region that interacts with or binds to the site-directed nuclease, e.g., Cas9. In the nucleic acid constructs provided herein, the constant region of an sgRNA can be from about 70 to 250 nucleotides in length, or about 75-100 nucleotides in length, 75-85 nucleotides in length, or about 80-90 nucleotides in length, or 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length. The overall length of the sgRNA can be, e.g., from about 80-300 nucleotides in length, or about 80-150 nucleotides in length, or about 80-120 nucleotides in length, or about 90-110 nucleotides in length, or, e.g, 90, 91, 92, 93, 94, 95, 96, 97, 98, 99, 100, 101, 102, 103, 104, 105, 106, 107, 108, 109, or 110 nucleotides in length.

It will be appreciated that it is also possible to use two-piece gRNAs (cr:tracrRNAs) in the present methods, i.e., with separate crRNA and tracrRNA molecules in which the target sequence is defined by the crispr RNA (crRNA), and the trans-activating crispr RNA (tracrRNA) provides a binding scaffold for the Cas nuclease.

In some embodiments, the sgRNAs comprise one or more modified nucleotides. For example, the polynucleotide sequences of the sgRNAs may also comprise RNA analogs, derivatives, or combinations thereof. For example, the probes can be modified at the base moiety, at the sugar moiety, or at the phosphate backbone (e.g., phosphorothioates). In some embodiments, the sgRNAs comprise 3′ phosphorothioate internucleotide linkages, 2′-O-methyl-3′-phosphoacetate modifications, 2′-fluoro-pyrimidines, S-constrained ethyl sugar modifications, or others, at one or more nucleotides.

The sgRNAs can be obtained in any of a number of ways. For sgRNAs, primers can be synthesized in the laboratory using an oligo synthesizer, e.g., as sold by Applied Biosystems, Biolytic Lab Performance, Sierra Biosystems, or others. Alternatively, primers and probes with any desired sequence and/or modification can be readily ordered from any of a large number of suppliers, e.g., ThermoFisher. Biolytic, IDT, Sigma-Aldritch, GeneScript, etc.

### RNA-Guided Nuclease

Any CRISPR-Cas nuclease can be used in the method, i.e., a CRISPR-Cas nuclease capable of interacting with a guide RNA and cleaving the DNA at the target site as defined by the guide RNA. In some embodiments, the nuclease is Cas9 or Cpfl. In particular embodiments, the nuclease is Cas9. The Cas9 or other nuclease used in the present methods can be from any source, so long that it is capable of binding to an sgRNA of the invention and being guided to and cleaving the specific CCR5 sequence targeted by the targeting sequence of the sgRNA. In particular embodiments, Cas9 is from Streptococcus pyogenes.

In addition to the CRISPR/Cas9 platform (which is a type II CRISPR/Cas system), alternative systems exist including type I CRISPR/Cas systems, type III CRISPR/Cas systems, and type V CRISPR/Cas systems. Various CRISPR/Cas9 systems have been disclosed, including Streptococcus pyogenes Cas9 (SpCas9), Streptococcus thermophilus Cas9 (StCas9), Campylobacter jejuni Cas9 (CjCas9) and Neisseria cinerea Cas9 (NcCas9) to name a few: Alternatives to the Cas system include the Francisella novicida Cpf1 (FnCpf1), Acidaminococcus sp. Cpf1 (AsCpf1), and Lachnospiraceae bacterium ND2006 Cpf1 (LbCpf1) systems. Any of the above CRISPR systems may be used to induce a single or double stranded break at the CCR5 locus to carry out the methods disclosed herein.

### Introducing the sgRNA and Cas Protein into Cells

The sgRNA and nuclease can be introduced into a cell using any suitable method, e.g., by introducing one or more polynucleotides encoding the sgRNA and the nuclease into the cell, e.g., using a vector such as a viral vector or delivered as naked DNA or RNA, such that the sgRNA and nuclease are expressed in the cell. In particular embodiments, the sgRNA and nuclease are assembled into ribonucleoproteins (RNPs) prior to delivery to the cells, and the RNPs are introduced into the cell by, e.g., electroporation. RNPs are complexes of RNA and RNA-binding proteins. In the context of the present methods, the RNPs comprise the RNA-binding nuclease (e.g., Cas9) assembled with the guide RNA (e.g., sgRNA), such that the RNPs are capable of binding to the target DNA (through the gRNA component of the RNP) and cleaving it (via the protein nuclease component of the RNP).

### Homologous Repair Templates

The CRISPR-Cas system typically includes a homologous repair template, or homologous donor template. The template includes a sequence that will be integrated into the genome in the place of a corresponding sequence in the genome, e.g., the sequence present between homologous regions in the template will replace a corresponding sequence present between the corresponding homologous regions in the genome. For example, in some embodiments the sequence in the template will introduce a deletion or an inactivating mutation into the genomic PTPN6 sequence, thereby eliminating or reducing SHP1 expression and/or activity in the cell. In particular embodiments, the sequence to be introduced is flanked in the template by PTPN6 homology regions, e.g., sequences of from, e.g., 100, 200, 300, 400, 500 or more nucleotides comprising homology to the genomic sequence on either side of the gRNA target sequence.

### d) Antibodies

In particular embodiments, the inhibitor is an anti-SHP1 antibody or an antigen-binding fragment thereof. In some embodiments, the antibody is a blocking antibody (i.e., an antibody that binds to a target and directly interferes with the target's function, e.g., SHP1 phosphatase activity). In some embodiments, the antibody is a neutralizing antibody (i.e., an antibody that binds to a target and negates the downstream cellular effects of the target). In particular embodiments, the antibody binds to mammalian SHP1 (e.g., human SHP1).

In some embodiments, the antibody is a monoclonal antibody. In some embodiments, the antibody is a polyclonal antibody. In some embodiments, the antibody is a chimeric antibody. In some embodiments, the antibody is a humanized antibody. In some embodiments, the antibody is a human antibody. In some embodiments, the antibody is an antigen-binding fragment, such as a F(ab′)2, Fab′, Fab, scFv, and the like. The term “antibody or antigen-binding fragment” can also encompass multi-specific and hybrid antibodies, with dual or multiple antigen or epitope specificities.

In some embodiments, an anti-SHP1 antibody comprises a heavy chain sequence or a portion thereof, and/or a light chain sequence or a portion thereof, of an antibody sequence disclosed herein. In some embodiments, an anti-SHP1 antibody comprises one or more complementarity determining regions (CDRs) of an anti-SHP1 antibody as disclosed herein. In some embodiments, an anti-SHP1 antibody is a nanobody, or single-domain antibody (sdAb), comprising a single monomeric variable antibody domain, e.g., a single VHH domain.

For preparing an antibody that binds to SHP1, many techniques known in the art can be used. See, e.g., Kohler & Milstein, Nature 256:495-497 (1975); Kozbor et al., Immunology Today 4:72 (1983): Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2nd ed. 1986)). In some embodiments, antibodies are prepared by immunizing an animal or animals (such as mice, rabbits, or rats) with an antigen for the induction of an antibody response. In some embodiments, the antigen is administered in conjugation with an adjuvant (e.g., Freund's adjuvant). In some embodiments, after the initial immunization, one or more subsequent booster injections of the antigen can be administered to improve antibody production. Following immunization, antigen-specific B cells are harvested, e.g., from the spleen and/or lymphoid tissue. For generating monoclonal antibodies, the B cells are fused with myeloma cells, which are subsequently screened for antigen specificity.

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Additionally, phage or yeast display technology can be used to identify antibodies and heteromeric Fab fragments that specifically bind to selected antigens (see, e.g., McCafferty et al., Nature 348:552-554 (1990); Marks et al., Biotechnology 10:779-783 (1992); Lou et al.m PEDS 23:311 (2010); and Chao et al., Nature Protocols, 1:755-768 (2006)). Alternatively, antibodies and antibody sequences may be isolated and/or identified using a yeast-based antibody presentation system, such as that disclosed in, e.g., Xu et al., Protein Eng Des Sel, 2013, 26:663-670; WO 2009/036379; WO 2010/105256; and WO 2012/009568. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can also be adapted to produce antibodies.

Antibodies can be produced using any number of expression systems, including prokaryotic and eukaryotic expression systems. In some embodiments, the expression system is a mammalian cell, such as a hybridoma, or a CHO cell. Many such systems are widely available from commercial suppliers. In embodiments in which an antibody comprises both a VH and VL region, the VH and VL regions may be expressed using a single vector, e.g., in a di-cistronic expression unit, or be under the control of different promoters. In other embodiments, the VH and VL region may be expressed using separate vectors.

In some embodiments, an anti-SHP1 antibody comprises one or more CDR, heavy chain, and/or light chain sequences that are affinity matured. For chimeric antibodies, methods of making chimeric antibodies are known in the art. For example, chimeric antibodies can be made in which the antigen binding region (heavy chain variable region and light chain variable region) from one species, such as a mouse, is fused to the effector region (constant domain) of another species, such as a human. As another example, “class switched” chimeric antibodies can be made in which the effector region of an antibody is substituted with an effector region of a different immunoglobulin class or subclass.

In some embodiments, an anti-SHP1 antibody comprises one or more CDR, heavy chain, and/or light chain sequences that are humanized. For humanized antibodies, methods of making humanized antibodies are known in the art. See, e.g., U.S. Pat. No. 8,095,890. Generally, a humanized antibody has one or more amino acid residues introduced into it from a source which is non-human. As an alternative to humanization, human antibodies can be generated. As a non-limiting example, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993): Jakobovits et al., Nature, 362:255-258 (1993); Bruggermann et al., Year in Immun., 7:33 (1993); and U.S. Pat. Nos. 5,591,669, 5,589,369, and 5,545,807.

In some embodiments, antibody fragments (such as a Fab, a Fab′, a F(ab′)2, a scFv, nanobody, or a diabody) are generated. Various techniques have been developed for the production of antibody fragments, such as proteolytic digestion of intact antibodies (see, e.g., Morimoto et al., J. Biochem. Biophys. Meth., 24:107-117 (1992); and Brennan et al., Science, 229:81 (1985)) and the use of recombinant host cells to produce the fragments. For example, antibody fragments can be isolated from antibody phage libraries. Alternatively, Fab′-SH fragments can be directly recovered from E. coli cells and chemically coupled to form F(ab′)2 fragments (see, e.g., Carter et al., BioTechnology, 10: 163-167 (1992)). According to another approach, F(ab′)2 fragments can be isolated directly from recombinant host cell culture. Other techniques for the production of antibody fragments will be apparent to those skilled in the art.

Methods for measuring binding affinity and binding kinetics are known in the art. These methods include, but are not limited to, solid-phase binding assays (e.g., ELISA assay), immunoprecipitation, surface plasmon resonance (e.g., Biacore™ (GE Healthcare, Piscataway, NJ)), kinetic exclusion assays (e.g., KinExA®), flow cytometry, fluorescence-activated cell sorting (FACS), BioLayer interferometry (e.g., Octet™ (FortéBio, Inc., Menlo Park, CA)), and western blot analysis.

### e) Peptides

In some embodiments, the inhibitor is a peptide, e.g., a peptide that binds to and/or inhibits the activity or stability of SHP1. In some embodiments, the inhibitor is a peptide that decreases SHP1 phosphatase activity. In some embodiments, the inhibitor is a peptide aptamer. Peptide aptamers are artificial proteins that are selected or engineered to bind to specific target molecules. Typically, the peptides include one or more peptide loops of variable sequence displayed by the protein scaffold. Peptide aptamer selection can be made using different systems, including the yeast two-hybrid system. Peptide aptamers can also be selected from combinatorial peptide libraries constructed by phage display and other surface display technologies such as mRNA display, ribosome display, bacterial display and yeast display. See, e.g., Reverdatto et al., 2015, Curr. Top. Med. Chem. 15:1082-1101.

In some embodiments, the agent is an affimer. Affimers are small, highly stable proteins, typically having a molecular weight of about 12-14 kDa, that bind their target molecules with specificity and affinity similar to that of antibodies. Generally, an affimer displays two peptide loops and an N-terminal sequence that can be randomized to bind different target proteins with high affinity and specificity in a similar manner to monoclonal antibodies. Stabilization of the two peptide loops by the protein scaffold constrains the possible conformations that the peptides can take, which increases the binding affinity and specificity compared to libraries of free peptides. Affimers and methods of making affimers are described in the art. See, e.g., Tiede et al., eLife, 2017, 6:e24903. Affimers are also commercially available, e.g., from Avacta Life Sciences.

### f) Vectors and Modified RNA

In some embodiments, polynucleotides providing SHP1 inhibiting activity, e.g., a nucleic acid inhibitor such as an siRNA or shRNA, or a polynucleotide encoding a polypeptide that inhibits SHP1 such as a blocking antibody fragment, are introduced into cells, e.g., dendritic cells, using an appropriate vector. Examples of delivery vectors that may be used with the present disclosure are viral vectors, plasmids, exosomes, liposomes, bacterial vectors, or nanoparticles. In some embodiments, any of the herein-described SHP1 inhibitors, e.g., a nucleic acid inhibitor or a polynucleotide encoding a polypeptide inhibitor, are introduced into cells, e.g., muscle cells, using vectors such as viral vectors. Suitable viral vectors include but not limited to adeno-associated viruses (AAVs), adenoviruses, and lentiviruses. In some embodiments, a SHP1 inhibitor, e.g., a nucleic acid inhibitor or a polynucleotide encoding a polypeptide inhibitor, is provided in the form of an expression cassette, typically recombinantly produced, having a promoter operably linked to the polynucleotide sequence encoding the inhibitor. In some cases, the promoter is a universal promoter that directs gene expression in all or most tissue types; in other cases, the promoter is one that directs gene expression specifically in dendritic cells.

### 3. Antigen Loading

Once mature DCs have been produced, they are loaded with antigen by culturing the cells in the presence of the antigen (or an antigenically active fragment thereof). For example, in some embodiments, DCs (e.g., BMDCs or DC2.4) are cultured (e.g., at a density of 2×10⁶ cells/ml) and treated with lipopolysaccharide (LPS) (e.g., at 100 ng/ml for 18 h) and then treated with an antigen (e.g., OVA protein at 250 μg/ml). In some embodiments, the DCs are incubated with the antigen for 24 hours in DC culture media supplemented with 10% exosome depleted FBS, which is prepared by the first ultracentrifugation at 100,000 g for 16 h, and then filtering using a 100 nm filter.

The antigen can be any antigen that corresponds to a tumor or infectious agent present in a subject, i.e., an antigen for which an increase in T cells specific for the antigen is desired. In some embodiments, the antigen corresponds to an antigen present on the surface of a tumor cell that is present in the subject, or an antigenically active fragment of the antigen. In some embodiments, the antigen corresponds to a tumor antigen peptide fragment comprising from 8 to 12 amino acid residues or from 15 to 24 amino acid residues, which are processed and presented by DCs to prime and activate specific T cells. In some embodiments, the antigen corresponds to an antigen from an infectious agent, e.g., virus or bacterium. In some embodiments, the antigen corresponds to an infectious disease antigen peptide fragment comprising from 8 to 12 amino acid residues or from 15 to 24 amino acid residues, which are processed and presented by DCs to prime and activate specific T cells.

In a particular embodiment, the DCs are loaded with an antigenic peptide such as intracellularly processed OVA-derived peptide (H-2Kb-SIINFEKL; SEQ ID NO:5). The antigen-bound H2-Kb molecules can be assessed when the DCs have or have not been treated with the SHP1 inhibitor, and inhibition will lead to an increase in the antigen in the membrane of BMDCs compared with control, and will further lead to the increased proliferation of antigen antigen-specific T cells.

### 4. Isolation of Extracellular Vesicles

Once the DCs in which SHP1 has been inhibited have been produced and loaded with antigen, extracellular vesicles are isolated from the DCs. As used herein, the term “extracellular vesicle” refers to a cell-derived vesicle comprising a membrane that encloses an internal space. Extracellular vesicles comprise all membrane-bound vesicles that have a smaller diameter than the cell from which they are derived. Generally, extracellular vesicles range in diameter from 20 nm to 1000 nm and can comprise various macromolecular cargo either within the internal space, displayed on the external surface of the extracellular vesicle, and/or spanning the membrane. The cargo can comprise nucleic acids, proteins, carbohydrates, lipids, small molecules, and/or combinations thereof. In particular embodiments, the present DC-derived extracellular vesicles (DC-EVs) have a size of about 30-150 nm and are initially formed within the cell by the inward budding of endosomal membranes. The molecular composition of DC-EVs harbor functional MHC-peptide complexes, tetraspanins, adhesion molecules, and costimulatory molecules that confer their immunomodulatory capabilities. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells. Extracellular vesicles can comprise different types of vesicles, including exosomes, ectosomes, microvesicles, microparticles, and large vesicles. In particular embodiments, the extracellular vesicles comprise exosomes.

Any suitable methods of isolating the exosomes (and/or other vesicles such as microvesicles) may be used. For example, an exosome isolation kit may be used. In some instances, isolating the exosomes may include filter sterilizing the enriched tissue extract through a series of filters. The series of filters may have sequentially reduced pore size. A final filter within the series of filters may have a pore size of 0.4 μm or less, 0.3 μm or less, 0.25 μm or less, 0.22 μm or less, 0.2 μm or less, 0.15 μm or less, or 0.1 μm or less. The pore size of the final filter may be selected such to isolate exosomes and/or microvesicles from the enriched tissue extract. In other cases, the exosomes and/or microvesicles may be isolated from the enriched tissue extract via magnetic cell separation or particle separation. In other cases, exosomes and microvesicles may be isolated from the extract via separation based on fluorescently labeled tags. In other instances, exosomes and microvesicles may be isolated based on hydrophobicity or affinity for vesicle membranes, or lack thereof.

In some embodiments, exosomes are purified from a DC cell culture by serial centrifugation, e.g., at 2,000 g and 10,000 g for 30 and 45 minutes, respectfully, to remove any cellular debris. Subsequently the culture is ultracentrifuged, e.g., at 150,000 g at 4° C. for two hours. In some embodiments, the pellet is washed, e.g., with PBS, and then centrifuged again at 150,000 g for 2 hours. The resulting pellet can then be frozen and stored, e.g., at −80° C., for subsequent analysis and/or use in the herein-described therapeutic methods.

In a particular embodiment, dendritic cells, e.g., bone-marrow derived dendritic cells (BMDCs), are cultured at a density of 2×10⁶ cells/ml and treated with LPS (100 ng/ml) for 18 h and then treated with OVA protein (250 μg/ml) for 24 hours in DCs culture media supplemented with 10% exosome depleted FBS, which is prepared by the first ultracentrifugation at 100,000 g for 16 h, and then filtering using a 100 nm filter. Supernatants are collected from 48-72 h DC cultures, and EVs are purified by a standard differential ultracentrifugation. In some embodiments, supernatants are subsequently subjected to sequential centrifugation steps at 300 g for 10 min to pellet whole cells, 2000 g for 10 min to remove dead cells, and 10,000 g for 30 min to discard cell debris. This resulting supernatant is then passed through a 0.22 μm syringe filter, and a pellet recovered at 100,000 g after 120 min of ultracentrifugation (SW32Ti rotor, Beckman Coulter). The supernatant is aspirated, and the pellet is resuspended in PBS (pH 7.3) and subsequently ultra-centrifuged at 100,000 g for another 120 min. Last, the exosome-containing pellet is dissolved in PBS. The purified exosomes are then analyzed and used for experimental procedures, e.g., microscopy, immunoblotting, FACS analysis, and in vitro functional assays, as well as in in vivo treatment of subjects, as described herein.

### C. Treatment

The present methods and compositions can be used to treat cancer, infectious disease, and other immune-related diseases, i.e., diseases for which an enhanced immune reaction can be beneficial, in subjects. In various embodiments, the subject may be an adult of any age, a child, or an adolescent. The subject may be male or female. In particular embodiments, the subject is a human.

Thus, in one aspect, provided herein is a method of enhancing anti-tumor immunity in a subject in need thereof, the method comprising administering to the subject dendritic cell (DC)-derived extracellular vesicles (DC-EVs), wherein the DC-EVs are obtained from dendritic cells that have been treated to inhibit src homologous region 2 domain-containing phosphatase-1 (SHP1) in the cells, and wherein the dendritic cells have been loaded with a tumor antigen corresponding to a tumor antigen known or believed to be present in the subject.

In another aspect, provided herein is a method of enhancing anti-infection immunity in a subject in need thereof, the method comprising administering to the subject dendritic cell (DC)-derived extracellular vesicles (DC-EVs), wherein the DC-EVs are obtained from dendritic cells that have been treated to inhibit src homologous region 2 domain-containing phosphatase-1 (SHP1) in the cells, and wherein the dendritic cells have been loaded with an infectious disease antigen corresponding to an infectious disease antigen known or believed to be present in the subject.

In some embodiments, the SHP1-modified DC-EVs are administered alone. In some embodiments, the DC-EVs are administered together with SHP1 -modified DCs. In some embodiments, SHP1-modified DCs are administered alone. In some embodiments, the DC-EVs and/or DCs are administered within a pharmaceutical formulation.

In some embodiments, the DCs and/or DC-EVs are administered to the subject in conjunction with another treatment such as immunotherapy and/or an anti-cancer or anti-infection agent.

In some embodiments, the subject has an infection and the DCs and/or DC-EVs are administered to the subject in conjunction with another appropriate therapy such as an anti-viral or antibiotic (anti-bacterial) compound.

In some embodiments, the subject has cancer, and the DCs and/or DC-EVs are administered to the subject as a combination therapy in conjunction with another anti-cancer therapy such as chemotherapy, radiation treatment, and/or surgical treatment. In some embodiments, the subject is administered one or more of a tyrosine kinase inhibitor, co-stimulatory mAb, epigenetic modulator, chemotherapeutic agent, radiation therapeutic, vaccine, adoptive T-cell therapeutic, or oncolytic virus.

In some embodiments, the subject receives surgical treatment for the cancer. For example, the patient may receive surgical resection (removal of the tumor with surgery). Small tumors may also be treated with other types of treatment such as ablation or radiation. Ablation is treatment that destroys tumors without removing them. These techniques can be used in patients with a few small tumors and when surgery is not a good option. They are less likely to cure the cancer than surgery, but they can still be very helpful for some people. Ablation is best used for tumors no larger than 3 cm across. For slightly larger tumors (1 to 2 inches, or 3 to 5 cm across), it may be used along with embolization. Because ablation often destroys some of the normal tissue around the tumor, it might not be a good choice for treating tumors near major blood vessels, the diaphragm, or major bile ducts. In some embodiments, the ablation is radiofrequency ablation (RFA). In some embodiments, the ablation is microwave ablation (MWA). In some embodiments, the ablation is cryoablation (cryotherapy). In some embodiments, the ablation is ethanol (alcohol) ablation, e.g., percutaneous ethanol injection (PEI).

In some embodiments, a patient with cancer is treated using radiation therapy. Radiation therapy uses high-energy rays, or particles to destroy cancer cells. Radiation can be helpful, e.g., in treating cancer that cannot be removed by surgery, cancer that cannot be treated with ablation or did not respond well to such treatment; cancer that has spread to areas such as the brain or bones; patients experiencing severe pain due to large cancers; and patients having a tumor thrombus.

In some embodiments, a patient with cancer is treated using drug therapy, e.g., targeted drug therapy, immunotherapy, or chemotherapy. Targeted drugs work differently from standard chemotherapy drugs and include, e.g., kinase inhibitors; Sorafenib (Nexavar), lenvatinib (Lenvima), Regorafenib (Stivarga), and cabozantinib (Cabometyx). Immunotherapy can comprise the administration of monoclonal antibodies. Monoclonal antibodies are designed to attach to a specific target. The monoclonal antibodies used to treat liver cancer affect a tumor's ability to form new blood vessels, also known as angiogenesis. These therapeutics are often referred to angiogenesis inhibitors and include: Bevacizumab (Avastin), which can be used in conjunction with the immunotherapy drug atezolizumab (Tecentriq); Ramucirumab (Cyramza).

Common chemotherapy drugs for treating cancer include, for example: Gemcitabine (Gemzar); Oxaliplatin (Eloxatin); Cisplatin; Doxorubicin (pegylated liposomal doxorubicin); 5-fluorouracil (5-FU); Capecitabine (Xeloda); Mitoxantrone (Novantrone), or combinations thereof. Chemotherapy can be regional when drugs are inserted into an artery that leads to the part of the body with the tumor, thereby focusing the chemotherapy on the cancer cells in that area of the body and reducing side effects by limiting the amount of drug reaching the rest of the body. For example, hepatic artery infusion (HAI), or chemo given directly into the hepatic artery, is an example of a regional chemotherapy that can be used for liver cancer.

In some embodiments, the subject receives immunotherapy in conjunction with the administration of the DCs and/or DC-EVs for the treatment of cancer, infection, or other immune-related condition. For example, in some embodiments the immunotherapy comprises administering an adoptive T-cell therapeutic (e.g., CAR-T cell, CAR-NK cell, CAR-Macrophage), co-stimulatory mAb, epigenetic modulator, vaccine against an infectious agent, tumor vaccine, oncolytic virus vaccine, TLR3/7/8/9 agonist, anti-CD47, or IL-2 receptor agonist to the subject. In some embodiments, the subject receives an immune checkpoint therapy (ICT). An important part of the immune system is its ability to keep itself from attacking normal cells in the body. To do this, it uses “checkpoints”—proteins on immune cells that need to be switched on or off to start an immune response. Cancer cells sometimes use these checkpoints to avoid being attacked by the immune system. Newer drugs that target these checkpoints hold a lot of promise as cancer (or infectious disease, or other immune-related condition) treatments and include, for example: PD-1 and PD-L1 inhibitors: Atezolizumab (Tecentriq), which can be used in conjunction with the targeted drug bevacizumab (Avastin); Pembrolizumab (Keytruda) and nivolumab (Opdivo), alone or in combination with ipilimumab (described below) may also be an option. In some embodiments, the immune checkpoint blockade binding agent is an anti-CTLA4, anti-PD1, anti-PD-L1, anti-LAG-3, anti-TIM-3, anti-TIGIT, anti-CD47 or anti-VISTA antibody.

### D. Pharmaceutical Compositions

In some embodiments, the herein-described SHP1-modified dendritic cells (DCs) and/or extracellular vesicles from the SHP1-modified dendritic cells (DC-EVs) are present within a pharmaceutical composition or formulation. The pharmaceutical compositions of the compounds of the present invention may comprise a pharmaceutically acceptable carrier. In certain aspects, pharmaceutically acceptable carriers are determined in part by the particular composition being administered, as well as by the particular method used to administer the composition. Accordingly, there is a wide variety of suitable formulations of pharmaceutical compositions of the present invention (see, e.g., REMINGTON'S PHARMACEUTICAL SCIENCES, 18TH ED., Mack Publishing Co., Easton, PA (1990)).

As used herein, “pharmaceutically acceptable carrier” comprises any of standard pharmaceutically accepted carriers known to those of ordinary skill in the art in formulating pharmaceutical compositions. Thus, the compounds, by themselves, such as being present as pharmaceutically acceptable salts, or as conjugates, may be prepared as formulations in pharmaceutically acceptable diluents: for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or the like, or as solid formulations in appropriate excipients.

The pharmaceutical compositions will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants (e.g., ascorbic acid, sodium metabisulfite, butylated hydroxytoluene, butylated hydroxyanisole, etc.), bacteriostats, chelating agents such as EDTA or glutathione, solutes that render the formulation isotonic, hypotonic or weakly hypertonic with the blood of a recipient, suspending agents, thickening agents, preservatives, flavoring agents, sweetening agents, and coloring compounds as appropriate.

The pharmaceutical compositions of the invention are administered in a manner compatible with the dosage formulation, and in such amount as will be therapeutically effective. The quantity to be administered depends on a variety of factors including, e.g., the age, body weight, physical activity, hereditary characteristics, general health, sex and diet of the individual, the condition or disease to be treated, the mode and time of administration, rate of excretion, drug combination, the stage or severity of the condition or disease, etc. In certain embodiments, the size of the dose may also be determined by the existence, nature, and extent of any adverse side effects that accompany the administration of a therapeutic agent(s) in a particular individual.

In certain embodiments, the dose of the compound may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

As used herein, the term “unit dosage form” refers to physically discrete units suitable as unitary dosages for humans and other mammals, each unit containing a predetermined quantity of a therapeutic agent calculated to produce the desired onset, tolerability, and/or therapeutic effects, in association with a suitable pharmaceutical excipient (e.g., an ampoule). In addition, more concentrated dosage forms may be prepared, from which the more dilute unit dosage forms may then be produced. The more concentrated dosage forms thus will contain substantially more than, e.g., at least 1, 2, 3, 4, 5, 6, 7, 8, 9, 10, or more times the amount of the therapeutic compound.

Methods for preparing such dosage forms are known to those skilled in the art (see, e.g., REMINGTON'S PHARMACEUTICAL SCIENCES, supra). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., REMINGTON'S PHARMACEUTICAL SCIENCES, supra).

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates (i.e., the parabens); pH adjusting agents such as inorganic and organic acids and bases; sweetening agents; and flavoring agents. The dosage forms may also comprise biodegradable polymer beads, dextran, and cyclodextrin inclusion complexes.

For oral administration, the therapeutically effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water. The lyophilized dosage form may further comprise a suitable vasoconstrictor, e.g., epinephrine. The lyophilized dosage form can be provided in a syringe, optionally packaged in combination with the buffer for reconstitution, such that the reconstituted dosage form can be immediately administered to an individual.

### E. Kits

In one aspect, kits are provided for the production of the herein-described DCs and DC-EVs and/or for the treatment of cancer, infection, or other condition in a subject. The kit may include, e.g., one or more agents for the production of DCs and DC-EVs, a container for holding, e.g., DCs, DC-EVs: and instructions for producing DCs and/or DC-EVs using the herein-described methods and/or administering the DCs and/or DC-EVs to a subject. The agents may be packaged in separate containers. The kit may further comprise one or more control reference samples and reagents for performing the herein-described methods. The kit may also comprise one or more devices or implements for carrying out any of the herein methods.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing instructions for methods of producing DCs and/or DC-EVs using the herein-described methods and/or administering the DCs and/or DC-EVs to a subject.

### EXAMPLES

The following examples are offered to illustrate, but not to limit, the claimed invention. Additional examples and figures can be found in Yuan et al., Vitamin E enhances cancer immunotherapy by reinvigorating dendritic cells via targeting checkpoint SHP1, Cancer Discovery 12:1742-59 (2022), which is incorporated herein in its entirety for all purposes.

### Example 1. Materials and Methods

The present example describes the materials and methods used for all of the other examples.

### Extracellular Vesicle (EVs) Purification and Characterization

To generate DC-EVs, BMDCs or DC2.4 were cultured at a density of 2×10⁶ cells/ml and treated with LPS (100 ng/ml) for 18 h and then treated with OVA protein (250 μg/ml) for 24 hours in DCs culture media supplemented with 10% exosome depleted FBS, which is prepared by the first ultracentrifugation at 100,000 g for 16 h, and then filtering using a 100 nm filter. Supernatants were collected from 48-72 h DCs cultures and EVs were purified by a standard differential ultracentrifugation. In brief, supernatants were subsequently subjected to sequential centrifugation steps at 300 g for 10 min to pellet whole cells, 2000 g for 10 min to remove dead cells, and 10,000 g for 30 min to discard cell debris. This resulting supernatant was then passed through a 0.22 μm syringe filter, and a pellet was recovered at 100,000 g after 120 min of ultracentrifugation (SW32Ti rotor. Beckman Coulter). The supernatant was aspirated, and the pellet was resuspended in PBS (pH 7.3) and subsequently ultra-centrifuged at 100,000 g for another 120 min. Last, the exosome-containing pellet was dissolved in PBS. The purified exosomes were then analyzed and used for experimental procedures. These exosomes were then subsequently used for microscopy, immunoblotting, FACS analysis, in vitro functional assays, and in vivo treatment of tumor-bearing mice, as described above.

For analysis by transmission electron microscopy, DC-EVs with the treatment of vehicle or VitE were fixed with 2% paraformaldehyde and placed on 100-mesh carbon-coated, formvar-coated nickel grids treated with poly-l-lysine for about 30 min. The grids were then stained with 2% phosphotungstic acid and imaged using a JEM 1010 transmission electron microscope (JEOL) at an accelerating voltage of 80 kV. Digital images were obtained using the AMT Imaging System (Advanced Microscopy Techniques Corp).

For FACS analysis, exosomes were conjugated with 4 μm aldehyde/sulfate latex beads (Cat. A37304, Thermo Fisher) by overnight incubation. The EVs-bound beads were incubated with glycine to block remaining binding sites and surface expression of SIINFEKL-loaded MHC-I complexes on DC-EVs was determined by staining with anti-mouse H-2K^{b} bound to SIINFEKL antibody. Data acquisition was conducted on BD Canto II Flow cytometer.

To verify the physical interactions between DC-EVs and CD8⁺ T cells, purified DC-EVs were stained with DiO (Cat. V22889, Thermo Fisher) in 100 μl PBS, and then washed with 10 ml PBS and pelleted by ultracentrifugation. For cell treatment, DC-EVs (10 μg/ml) (unless specified otherwise) were used on the basis of protein concentration determined by a BCA Protein Assay Kit (Pierce). Unstimulated mouse CD8⁺ OT-1 T cells (2×10⁵ cells/well in 96-well plates) were treated with DiO-labelled DC-EVs for 2 h, and then fixed for FACS or confocal microscopy after immunostaining for CD8⁺ T cells with anti-mouse CD8a and anti-mouse H-2Kb bound to SIINFEKL Ab. To assay for the proliferation of CD8⁺ T cells, 1×10⁶ CD8⁺ OT-1 T cells were stained with CFSE (Cat.423801, BioLegend) at 5 μM. CFSE-labeled CD8⁺ OT-1 T cells were treated with OVA-loaded vehicle-DC-EVs or VitE-DC-EVs generated as described above. After 72 h, the T-cell proliferation was analyzed by FACS. Unstimulated CFSE-labelled cells served as a non-dividing control. IFN-γ levels in culture supernatant were measured with mouse IFN-y ELISA kit (Cat. 430807, BioLegend) according to the manufacturer's instructions.

### Tumor Challenge and Treatment Experiments

For the mammary tumor models, 2×10⁵ EMT6, 4T1, or E0771 cells were orthotopically injected into the mammary fat pad (MFP) of female BALB/cJ mice (4T1, EMT6) or C57BL/6 (E0771) on day 0. For the melanoma tumor challenges, 2×10⁵ B16-F10, B16-GMCSF, or B16-FLT3L cells were subcutaneously (s.c.) injected into the right flank of C57BL/6J mice on day 0. Syngeneic orthotopic PDAC tumors were established by surgical implantation. Briefly, 2,000 HY19636 KPC cells in 50 μl PBS were injected into each pancreas of the C57BL/6J mouse.

Treatments were given as single agents or in combinations with the indicated regimens for each group. VitE (D-α-Tocopherol type VI, Cat.T1539, Sigma-Aldrich) was formulated in the vehicle (10% ethanol in sterile water) and administered by oral gavage once a day at a dose of 50 mg/kg body weight (100 μl at 10 mg/ml per day), corresponding to 400 to 600 mg/day in a human weighing 60 kg, which is within the range of doses recommended for supplement and pharmacological use. VitE treatment was initiated on day 4 ending until the ending point. The SHP1 inhibitor TPI (Cat. 22480, Cayman) was administered by oral gavage once a day at 3 mg/kg body weight. Treatment was initiated on day 4 ending on day 20 post-inoculation. Control groups received vehicle (PBS) without the active product.

For immune checkpoint blockade (ICB) experiments, mice were given by intraperitoneal (i.p.) injection of anti-mouse PD-1 antibody (200 μg, clone RMP1-14, generated from hybridoma or purchased from BioXcell, Cat. BE0164) and/or anti-mouse CTLA-4 antibody (100 μg, clone 9D9, Cat. BE0164, BioXcell) on days 7, 10, 13 and 16 for the indicated tumor models. Rat IgG2a isotype control (200 μg, clone 2A3, BE0089) and/or mouse IgG2b isotype control (100 μg, clone MPC-11, BE0086) respectively was used in control mice corresponding to the ICB treatment group.

For in vivo DCs adoptive transfer experiments, 2×10⁵ B16-GMCSF tumor cells mixed with sgCtrl-DC2.4 or sgPtpn6-DC2.4 cells at a ratio of 1:1 were injected subcutaneously into C57BL/6J mice, respectively. For DC-EVs treatment experiments, 30 μg DC-EVs was administrated intravenously (i.v.) on day 1, 7, 14 after tumors transplantation.

Where indicated, mice were vaccinated subcutaneously on their contralateral flank with either PBS or 1.0×10⁶ 35 Gy-irradiated GM-CSF-secreting B16 cells or GM-CSF-secreting 4T1 cells (GVAX) on days 1, 4, and 7 post-tumor injections.

For chemo combination treatment, HY19636 KPC tumor-bearing mice were treated with gemcitabine (GEM) at 25 mg/kg body weight by i.p. injection once a week from day 7 and 4T1 TNBC tumor-bearing mice were given with doxorubicin (Dox, 5 mg/kg body weight) by i.v. once a week from day 10. The chemotherapeutic doses and schedules used in this study are below or within the range of the concentrations used in the clinic after converting drug doses between species.

For tumor volume analysis, each tumor was measured every 3-4 days with a digital caliper beginning on day 7 after the challenge. Tumor volume was calculated using the formula V=(L×W²)/2 and expressed as mm³, where V is tumor volume, L is the length of the tumor (longer diameter) and W is the width of the tumor (shorter diameter). Mice were monitored for tumor growth and survival. Measurements were assessed manually by assessing the longest dimension (length, L) and the longest perpendicular dimension (width, W). Tumor volume was calculated using the formula: (L×W²)/2.

### Human Monocyte-Derived Dendritic Cells (MoDCs) Generation and Cultures

Human leukocyte concentrate (buffy coat) was collected from healthy blood donor volunteers by MD Anderson Blood Donor Center. Written informed consent was obtained from all participating blood donors and the use of anonymized leftover specimens for scientific purposes was approved by the Ethics Committee of the MD Anderson Cancer Center. For the generation of monocyte-derived dendritic cells (MoDCs), peripheral blood of healthy individuals was applied to a Ficoll gradient (BD), and mononuclear cells were freshly isolated and cultured in the dish in a humidified incubator for 1 hour to let monocytes adhere. After removing the medium with non-adherent cells, the adherent monocytes were collected through vigorous pipetting. For differentiation of monocytes into MoDCs, 0.5×10⁶ cells/mL purified monocytes were cultured in DC culture medium (RPMI 1640 containing 2 mM L-glutamine and 25 mM HEPES, supplemented with 10 mM Sodium Pyruvate. 1% MEM nonessential amino acids, 100 U/ml penicillin/streptomycin, 50 μM 2-mercaptoethanol, and 10% FBS) supplemented with 800 U/mL of human GM-CSF (Cat.300-03, PeproTech) and 500 U/mL human IL-4 (Cat.200-04, PeproTech), and the media were refreshed with supplement cytokines for every 3 days. After 7-day incubation, when the majority of monocytes differentiated to immature DCs, cells were re-cultured in fresh DCs culture medium supplemented with 50 ng/ml tumor necrosis factor-alpha (TNF-α) (Cat.300-01A, PeproTech) for an additional one days to generate mature DCs.

### Human T Cell Isolation

Healthy donor buffy coats were collected by MD Anderson Blood Donor Center. Primary human T cells were isolated using the RosetteSep human T cell enrichment cocktail (Cat. 15061, Stem Cell Technologies) according to the manufacturer's protocol. Isolated T cells were cryopreserved at 1×10⁷ cells per vial in a cryopreservation medium. Autologous T cells were used for coulure with MoDCs.

### Bone Marrow-Derived Dendritic Cells (BMDCs) Generation and Cultures

Bone marrow (BM) was harvested from C57BL/6J, BALB/Cj mice by flushing femurs, tibias, and humeri with DCs culture medium. Bone-marrow cells were strained through a 70-μm filter (BD) and centrifuged before resuspension in RBC lysis buffer (150 mM NH₄Cl/10 mM NaHCO3/1 mM EDTA) for 5 min on ice. The remaining cells were plated at 2×10⁶/ml in DCs culture medium supplemented with 20 ng/ml of mGM-CSF (Cat. AF-315-03, PeproTech) and 10 ng/mL mIL-4 (Cat.214-14, PeproTech), and the media were refreshed with supplement cytokines for every 3 days. After 7-day incubation, the majority of BM cells (>90%) were differentiated to DCs. BMDCs were re-cultured in fresh DCs culture medium supplemented with lipopolysaccharide (LPS, Cat.tlrl-eblps, InvivoGen) for an additional one days to generate mature DCs. Where indicated, BMDCs were treated with vehicle or VitE at the indicated concentration for 2 days. In some instances, BMDCs were pre-incubated in control (vehicle) or TCM for 12 hours.

### Silencing SHP1 in DCs

MISSIONR esiRNA obtained from the Sigma-Aldrich (Cat. EMU084951) were used for targeting mouse Ptpn6 in mouse BMDCs. MISSION® esiRNA obtained from the Sigma-Aldrich (Cat. EHU066321) was used for targeting human Ptpn6 in human MoDCs. To generate DC2.4 Ptpn6-knockout cells, pLentiCRISPRv2 plasmids from Addgene (Cat #52961) were used following the standard protocol published by Feng Zhang's lab. Three guide RNA (sgRNA) sequences targeting Pipn6 (sgRNA-Ptpn6) were obtained from the Genome-wide gRNA databases for CRISPR genome editing and transcription activation. The target sequence of each gRNA is:

Ptpn6-sgRNA1:
(SEQ ID NO: 1)
CAAACTTCTCCCCTCCGTAC
Ptpn6-sgRNA2:
(SEQ ID NO: 2)
GACTTCTATGACCTGTACGG
Ptpn6-sgRNA3:
(SEQ ID NO: 3)
CTGGCCCAGGTTCCCCGCTC
Non-targeting sgRNA:
(SEQ ID NO: 4)
GCGAGGTATTCGGCTCCGCG

All plasmids with target sequence inserted were sequenced by the Sequencing and Microarray Facility at MD Anderson before being packaged into virus in HEK293 cells. The supernatant of culture media containing the lentiviral particles of respective target DNA sequences is used to transduce fresh DC2.4 cells. After puromycin selection, DNA of stably transduced DC2.4 cells was extracted for confirmation of gene editing by sanger sequencing. And the loss of SHP1 protein expression was assayed by Western blotting.

### Molecular Docking VitE Into Inactive SHP1

High-resolution crystal structures of auto-inhibitory SHP1 (PDB ID: 2B30) were obtained from Protein Data Bank. 3D structure of VitE (αT) was downloaded from PubChem (PubChem CID: 14985). The SHP1 structure and VitE structure were first uploaded to Webina—an AutoDock-based webserver (durrantlab.pitt.edu/webina). For all the top 9 docking complex models, VitE is bound in its central cavity formed at the interface of all three domains of SHP1 (N-SH2, C-SH2, and PTP domains). The model with the highest binding affinity was selected for detailed analysis.

### SHP1 Phosphatase Assay

We assessed the phosphatase activity of SHP1 by using the RediPlate 96 EnzChek Tyrosine Phosphatase Assay Kit (ThermoFisher). We immunoprecipitated SHP1 from the vehicle or VitE-treated DC2.4 cells or BMDCs with a monoclonal SHP1 antibody prebound to protein G Sepharose beads. After a 2 h incubation, we washed beads three times with PBS containing 1% Igepal CA-630 and 5 mM dithiothreitol (DTT). We placed beads into RediPlate wells and incubated them for 30 min at 25° C. before reading them for fluorescence. For the inhibition assay, indicated dosage of VitE was placed in 96-well plates and mixed with recombinant SHP1 protein (0.05 μg/well) in reaction buffer. The plates were incubated at room temperature for 30 min and then aspirate the mixture into RediPlate wells to initiate

PTP reaction and incubated for 30 min in darkness at 25° C. The fluorescence signal was monitored using a microplate reader (BioTek) using excitation and emission wavelengths of 342 nm and 458 nm, respectively. They have compared with that of control SHP1 PTP reaction in the absence of any compound (100%) for calculating relative SHP1 inhibition induced by VitE after subtracting the background signal of the substrate. The inhibitor dose-response curves were analyzed using normalized IC50 regression curve fitting with control-based normalization.

### Example 2. Natural VitE Enhances Cancer Immunotherapy in Humans and Mice

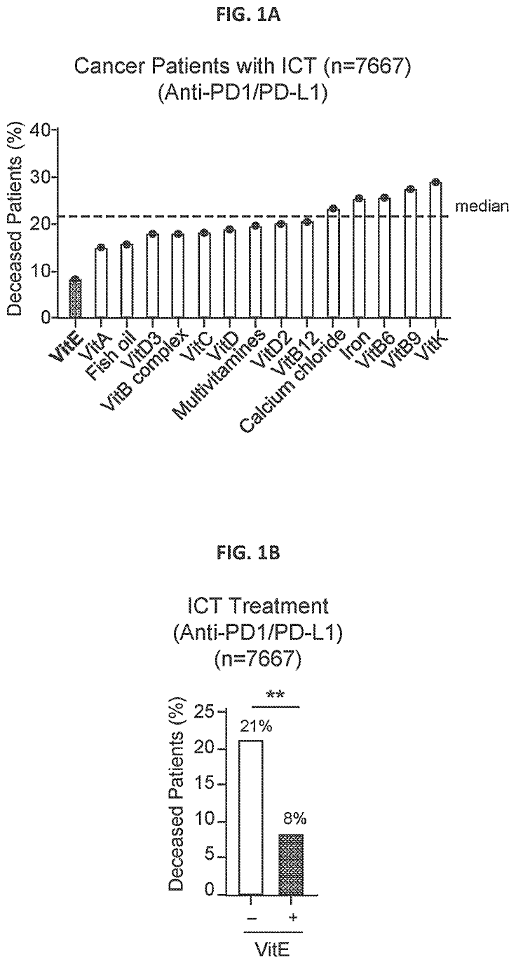

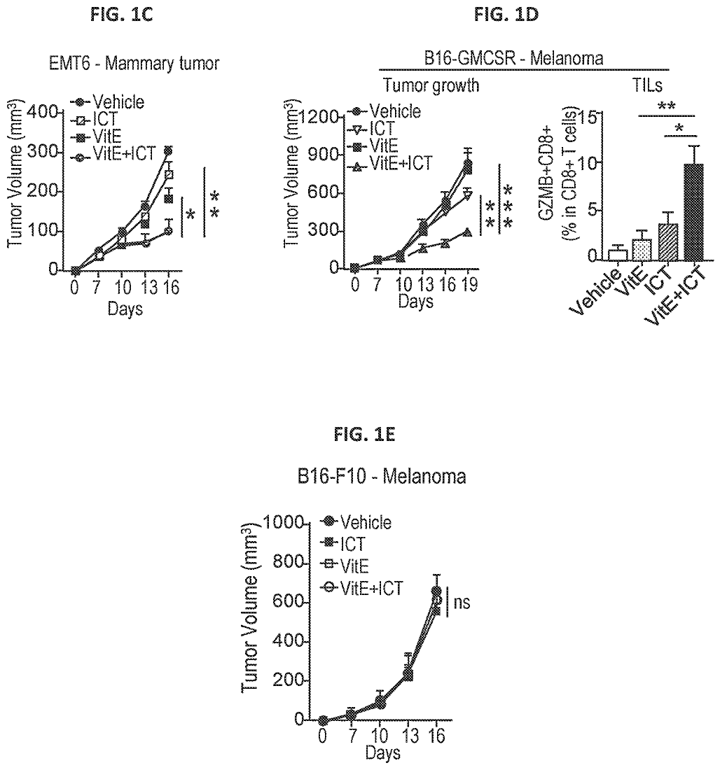

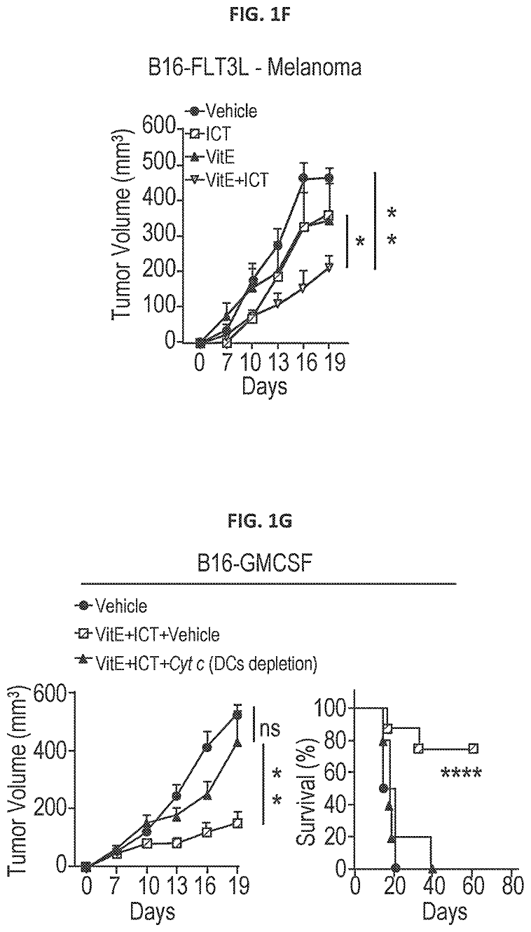

We retrospectively analyzed electronic health records for the clinical outcomes of cancer patients receiving ICT immunotherapies (anti-PD-1/PD-L1). We found that cancer patients who took VitE during anti-PD-1/PD-L1 treatments had significantly improved survival rate than patients taking any of the other 14 common dietary or nutritional supplements (FIGS. 1A-1B). Taking VitE while receiving ICT was associated with a trend of improved overall survival in breast cancer, melanoma, and prostate cancer patients. Consistently, VitE and ICT combo-treatment achieved significantly improved therapeutic response in mice bearing the EMT6 mammary tumors (FIG. 1C) and the B16-GMCSF melanoma (FIG. 1D).

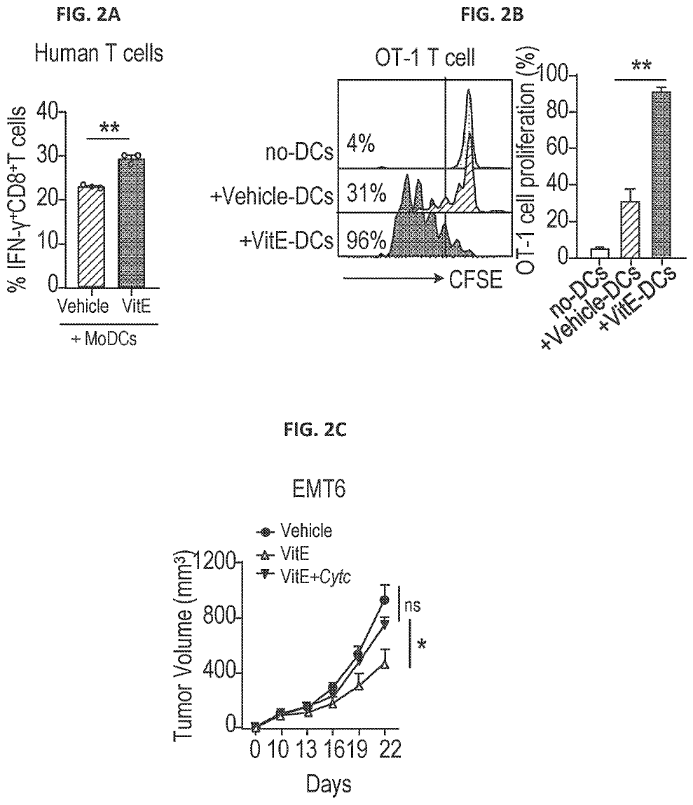

When testing in mice bearing the B16-F10 melanoma, we found that VitE and ICT combo-treatments showed no therapeutic effect (FIG. 1E), unlike in the B16-GMCSF melanoma (FIG. 1D), which were generated from the B16-F10 cell line by overexpressing the granulocyte-macrophage colony-stimulating factor (GM-CSF) that is positively associated with DCs infiltration (De Henau et al., 2016). Likewise, in mice bearing the B16-FLT3L melanoma, a B16-F10 subline overexpressing FLT3L that is known to promote hematopoietic progenitor commitment to the DC lineage (Karsunky et al., 2003), VitE effectively enhanced their ICT responses (FIG. 1F). These results suggest that VitE-mediated ICT response enhancement highly depends on DCs infiltration. Indeed, depletion of DCs by cytochrome c (Cyt c) (Lin et al., 2008) abolished the VitE-augmented ICT response in the B16-GMCSF model (FIG. 1G). We injected EMT6 cells into mammary fat pad (MFP) of BALB/c mice and treated them with VitE and found reduced tumor growth compared with the vehicle-treated group (n=10 per group: average tumor volume at day 15: vehicle-treated, 350 mm³; VitE-treated, 173 mm³). Also, DC-depletion abolished the VitE effect in EMT6 tumor-bearing mice (FIG. 2C), confirming the DCs-dependency of VitE effect in vivo. Therefore, VitE administration enhances immunotherapy dependent on DCs infiltration to provoke antitumor T-cell immunity.

To examine the association between DC activation and antitumor T-cell immunity in patients, we performed bioinformatics analysis of human breast cancer data in The Cancer Genome Atlas. We found that pembrolizumab (anti-PD-1)-treated patients with melanoma with higher tumor-infiltrating aDCs had markedly increased OS compared with patients with lower aDC infiltration (PRJEB23709; ref. 18:;data not shown: see U.S. Provisional Application No. 63/252,721, FIG. 2J; see also Yuan et al. 2022, FIG. 2I).

### Example 3. Natural VitE Activates DCs Function as an Inhibitor of DC-Intrinsic Checkpoint SHP1

Notably, we validated that VitE treatment enhanced the activation of human monocyte-derived DCs (MoDCs), as indicated by significantly increased expression of DC activation markers CD40, CD86, HLA-DR. We assessed the expression of co-stimulatory molecules on human MoDCs treated with indicated dietary components and nutritional supplements including VitE (50 μg/ml). VitC (20 μg/ml), VitD (100 ng/ml), VitB (folate, 200 ng/ml), VitB2 (2 μg/ml), VitB12 (10 ng/ml), and DHA (Omega-3, 100 μM) for 48 h, and then stimulated with LPS (100 ng/ml) for 24 h. The mean fluorescence intensity (MFI) of each molecule was normalized to the MFI of the vehicle-treated group measured by flow cytometry. We then quantitated DC activation markers CD40, CD86, and HLA-DR on human MoDCs treated with vehicle or VitE (50 μg/ml) for 48 h in the presence of LPS (100 ng/ml), as calculated by relative mean fluorescence intensity (MFI) compared to controls. Compared with other common nutritional supplements, VitE most effectively activated DCs (data not shown; see Yuan et al. 2022, FIG. 2A; see also U.S. Provisional Application No. 63/252,721, FIG. 2A), as indicated by the significantly increased expression of CD40, CD86, and HLA-DR (data not shown; see Yuan et al. 2022, FIG. 2B: see also U.S. Provisional Application No. 63/252,721, FIG. 2B).

Functionally, VitE-treated human MoDCs increased autologous IFNγ+CD8+ cytotoxic T cells (FIG. 2A). Furthermore, VitE-treated and ovalbumin (OVA)-loaded BMDCs increased the OVA-derived SIINFEKL antigen-specific OT-1 T cells in coculture (FIG. 2B).

Cytometry by time-offlight (CyTOF) analysis of CD45+ immune cells isolated from EMT6 tumors showed that VitE treatment increased the tumor infiltrations of CD11c+DCs and CD8+ T cells (data not shown: see Yuan et al. 2022, FIG. 2E; see also U.S. Provisional Application No. 63/252,721, FIG. 2E). Further analyses of CD11c+DCs from EMT6 tumors showed that VitE treatment induced a striking component shift of tumor-associated DCs (TADC) from naïve MoDCs (CD14+CD206+) and plasmacytoid DCs (pDC; CD11cloB220+) to conventional DCs (CD103+cDC1 and CD11b+cDC2) (data not shown; see Yuan et al. 2022, FIG. 2F: see also U.S. Provisional Application No. 63/252,721, FIG. 2F;), which are critical for tumor-reactive T-cell responses, indicating that VitE treatment instigated a functional differentiation of the DC compartments and induced polarization to cross-presenting DCs. Consistently, the majority of VitE induced DCs exhibited the characteristics of activated DCs (CD80+CD86+MHC-II+Ki-67+; data not shown; see Yuan et al. 2022, FIG. 2G; see also U.S. Provisional Application No. 63/252,721, FIG. 2G). Significantly. TADCs of VitE-treated tumors promoted T-cell proliferation more than TADCs from vehicle-treated tumors (data not shown; see Yuan et al. 2022, FIG. 2H; see also U.S. Provisional Application No. 63/252,721, FIG. 2H). Therefore, VitE-induced enhancement of DC function could adequately trigger antitumor T-cell immunity in vivo.

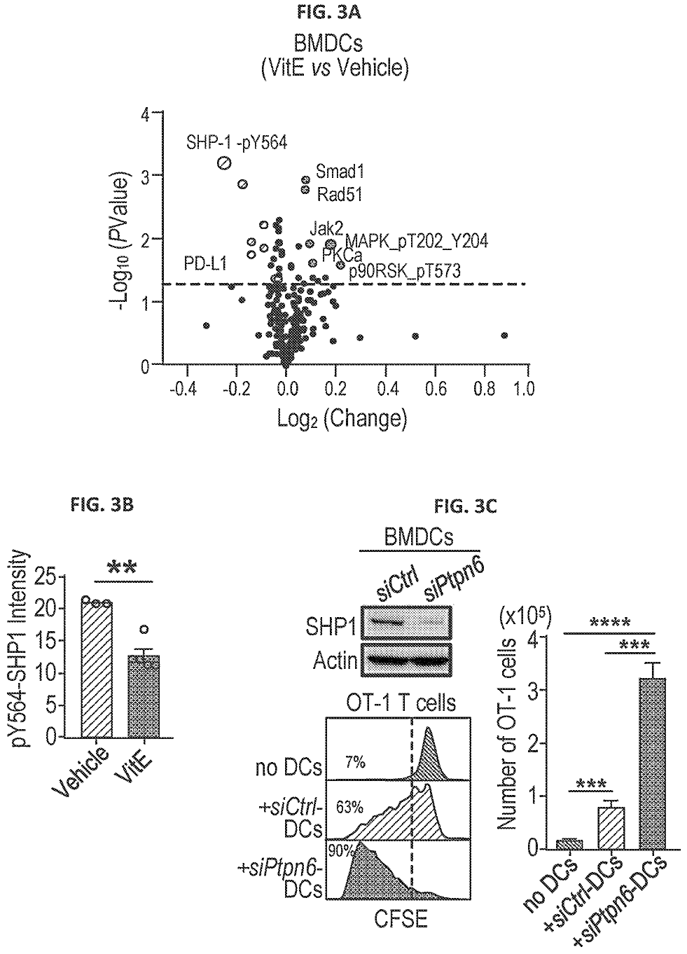

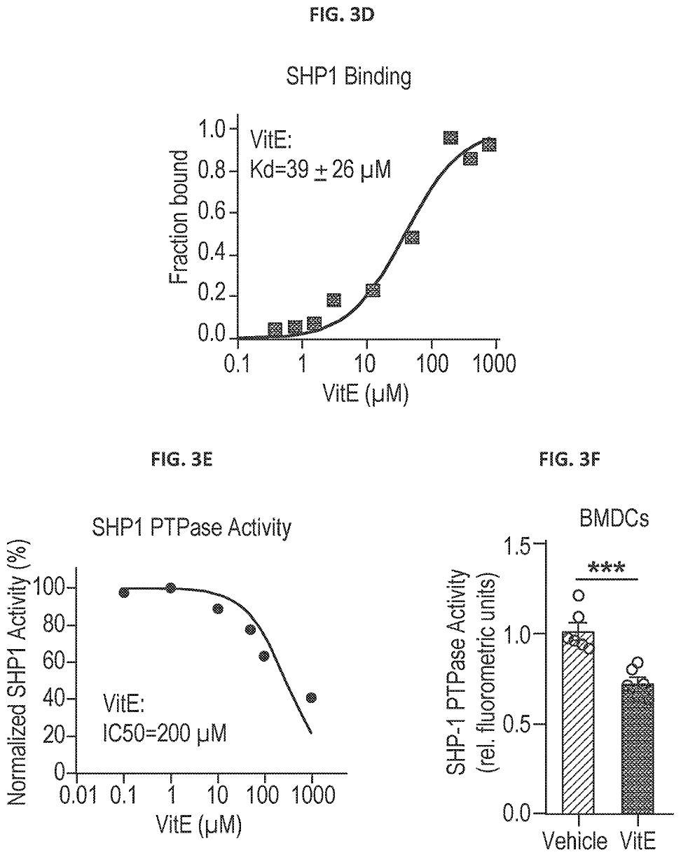

To explore the molecular mechanism of VitE on DCs function, we performed a reverse-phase protein array (RPPA) analysis on DCs. We found that VitE treatment decreased the phosphorylation at tyrosine 564 (Y564) of the Src homologous region 2 domain-containing phosphatase-1 (SHP1) in DCs (FIG. 3A). The pY564-SHP1 levels were markedly higher in TADCs than in splenic DCs of EMT6 tumor-bearing mice (data not shown: see Yuan et al. 2022. FIG. 3B: see also U.S. Provisional Application No. 63/252,721, FIG. 3B). VitE treatment reduced the pY564-SHP1 level in TADCs (FIG. 3B) and increased MAPK pathway activation, correspondingly. SHP1, a cellular tyrosine phosphatase encoded by the Ptpn6 gene, has been shown to be a DC-intrinsic checkpoint in vitro (Carmi et al., 2016) and behaves as a negative regulator in cellular signaling pathways. These findings indicated that VitE treatment activated DCs function by inhibiting SHP1.

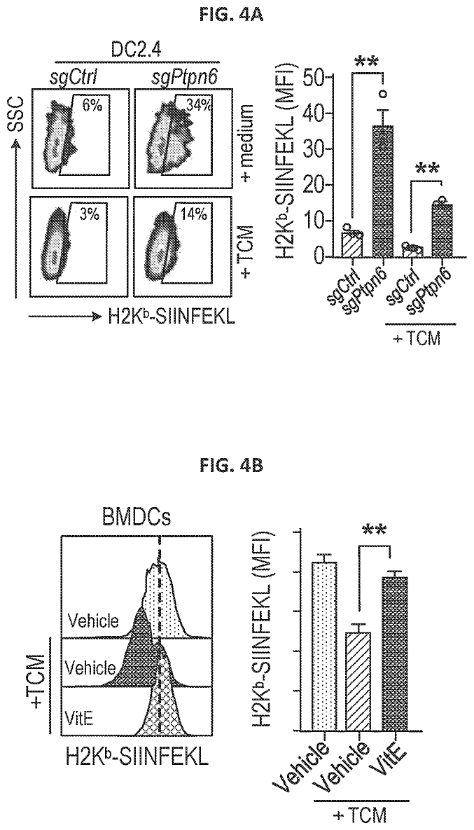

To computationally assess the implicated interaction between VitE and SHP1 at the atomic, we performed molecular docking analysis and found that VitE preferentially docked in the central cavity. at an interface formed by the N-terminal SH2 (N-SH2) and C-terminal SH2 (C-SH2) domains with the protein tyrosine phosphatase (PTP) domain of SHP1 (data not shown: see Yuan et al. 2022, FIG. 4A; see also U.S. Provisional Application No. 63/252,721. FIG. 3E and FIG. 3F). This region is critical for stabilizing SHP1 auto-inhibitory conformation through a steric N-SH2 domain block of the phosphatase active (Yang et al., 2003), indicating VitE function as an allosteric inhibitor of SHP1. In detail, three types of molecular forces are implied in the interaction between folded VitE and all three domains of inactive SHP1: 1) hydrogen bonds with His112 (C-SH2) and Ser250 (PTP); 2) hydrophobic interactions with the sidechains of His112 (C-SH2), Tyr214 (PTP) and Glu247 (PTP); and 3) intramolecular hydrogen bonds: etheric oxygen from the VitE chroman moiety receives a hydrogen bond from Ser 250 (PTP), which is intramolecularly engaged with Ser107 (N-SH2) (data not shown; see Yuan et al. 2022, FIG. 4B: see also U.S. Provisional Application No. 63/252,721, FIG. 3F). To experimentally measure the VitE-SHP1 interaction to gain further mechanistic understanding of the effect of VitE (α-tocopherol) on SHP1 activity, we employed an in vitro binding assay that revealed a direct interaction between SHP1 and VitE with a dissociation constant of 39 μM (FIG. 3D). Functionally, VitE demonstrated moderate inhibitory tyrosine phosphatase activity (IC50=50-200 μM) against SHP1 immunoprecipitated from LPS-activated DC2.4 cells (FIG. 3E). Determination of in vivo SHP1 activity in BMDCs treated with vehicle or VitE upon the LPS stimulation confirmed the marked reduction of SHP1 function in the VitE-treated BMDCs (FIG. 3F). Cumulatively, our computational structural analysis and biochemical assays revealed VitE binds SHP1 and inhibits SHP1 phosphatase activity.

### Example 4. VitE Enters DCs Through SCARB1 to Inhibit SHP1

VitE can enter enterocytes via the SCARB1 receptor, and SCARB1 facilitates the uptake of cholesteryl esters from high-density lipoproteins. Notably, DCs have the highest SCARB1 expression among all major human immune cell populations (data not shown: see Yuan et al. 2022, FIG. 4F; see also U.S. Provisional Application No. 63/252,721, FIG. 4A). To determine whether SCARB1 is required for VitE-induced DC activation, we generated BMDCs from Scarb1-/-versus wild-type mice. VitE treatment failed to inhibit pY564-SHP1 in Scarb1-/-DCs unlike in wild-type DCs (data not shown: see Yuan et al. 2022, FIG. 4G; see also U.S. Provisional Application No. 63/252,721, FIG. 4B). Additionally, VitE-treated Scarb1-/-DCs did not induce proliferation of cocultured T cells, in contrast to VitE-treated wild-type DCs (data not shown: see Yuan et al. 2022. FIG. 4H; see also U.S. Provisional Application No. 63/252,721, FIG. 4C). Taken together, VitE enters DCs via SCARB1 and binds SHP1 to inhibit pSHP1, leading to DC activation, which increases antitumor T-cell immunity (data not shown; see Yuan et al. 2022, FIG. 4I; see also U.S. Provisional Application No. 63/252,721, FIG. 4D).

The mean tumor volume of intradermal B16-GMCSF implants in C57BL/6 mice treated with vehicle or TPI-1 (3 mg/kg by oral administration, daily; n=5 per group) was assessed, with TP1-1 treated mice having significantly smaller tumor volume (mm3) by day 19 of treatment. Flow cytometric analysis of OT-1 proliferation and counting of OT-1 cell numbers (n=3) under cocultured with sgCtrl-DC2.4 or sgPipn6-DC2.4 cells loaded with OVA (250 μg/ml) was performed. Exemplary FCA numbers were 52% sgCtrl-D2.4 and 67% sgPtpn6-DC2.4 and exemplary OT-1 cell counts were approx. 20-30% sgCCtrl and approx. 50-70% sgPtpn6 (data not shown: see U.S. Provisional Application No. 63/252,721, FIG. 4F). Tumor growth curves of B16-GMCSF tumor cells alone, and co-implanted with sgCtrl-or sgPtpn6-DC2.4 cells in C57BL/6 mice (data not shown; see Yuan et al. 2022, FIG. 3I; see also U.S. Provisional Application No. 63/252,721. FIG. 4G).

### Example 5. Targeting SHP1 Enhances Antigen Presentation by DCs and DC-EVs

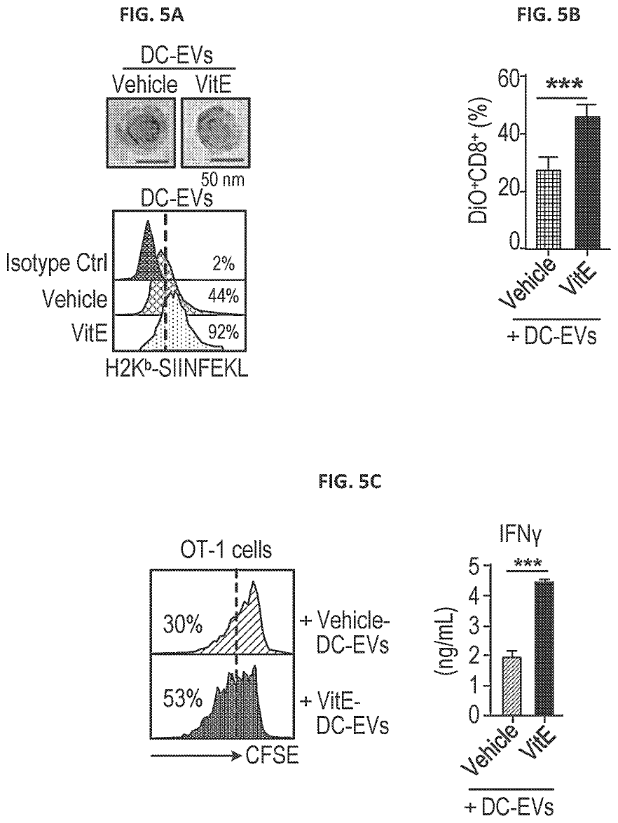

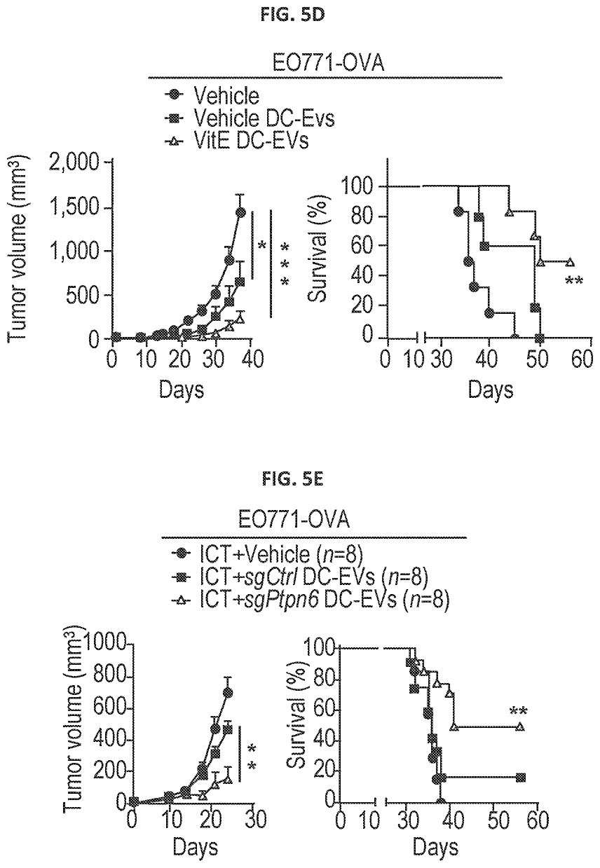

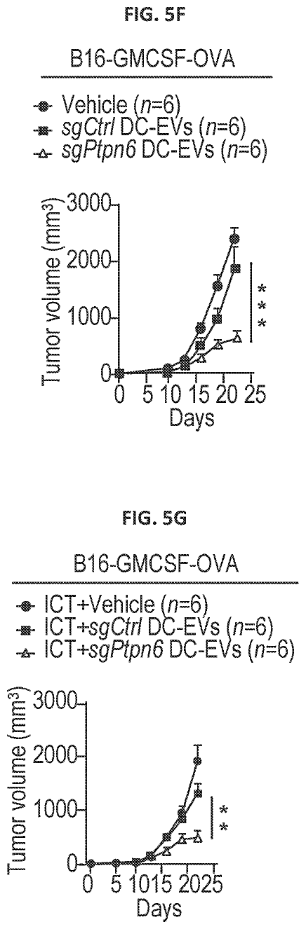

The most critical function of DCs is the cross-presentation of tumor antigens to T-cells (Guermonprez et al., 2002). We assessed the membrane MHC-I complexes that were loaded with intracellularly processed OVA-derived peptide (H-2Kb-SIINFEKL) on OVA-loaded DCs with VitE-treatment. SIINFEKL-bound H2-Kb molecules were significantly increased on the membrane of BMDCs treated with VitE compared with control (FIG. 4B), which lead to the increased proliferation of SIINFEKL antigen-specific OT-1 T cells (FIG. 2B). Therefore, VitE administration enhances the activation and antigen cross-presentation function of DCs to provoke antigen-specific antitumor T-cell immunity. We quantified H-2Kb-SIINFEKL in the vehicle-or VitE-treated sgCtrl-DC2.4 or sgPtpn6-DC2.4 (n=3). Of note, both VitE treatment and SHP1 knockout significantly increased H-2Kb-SIINFEKL molecules on DC2.4 cells compared with vehicle-treated sgCtrl-DC2.4 cells, but VitE treatment did not further increase H-2Kb-SIINFEKL molecules on sgPtpn6-DC2.4 cells (data not shown; see Yuan et al. 2022, FIG. 5F; see also U.S. Provisional Application No. 63/252,721, FIG. 5C). Thus. SHP1 inhibition in DCs by VitE treatment or Ptpn6 knockout enhances antigen cross-presentation that boosts CD8+ T-cell antitumor immunity. We also showed that deleting the SHP1 -encoding Ptpn6 gene by CRISPR-Cas9 in DC2.4 murine DC cell line (FIG. 3C). SIINFEKL-bond H2-Kb molecules were significantly increased on the membrane of sgPtpn6-DC2.4 cells compared with sgCtrl-DC2.4 cells (FIG. 4A). Mouse siPtpn6-BMDCs induced OT-1 T-cell proliferation significantly more than siCtrl-BMDCs under co-culture (FIG. 3C), like the effect of VitE treatment (FIG. 2B).

To explore the potential mechanism of enhanced cross-presentation in SHP1-inhibited DCs, BMDCs transfected with scrambled siRNA (control) and Ptpn6 siRNA were labeled and analyzed by confocal microscopy. We observed a remarkable reduction of phagolysosome fusion after silencing SHP1 in BMDCs, as indicated by reduced staining of lysotracker and LAMP1 (data not shown: see Yuan et al. 2022, FIG. 5G; see also U.S. Provisional Application No. 63/252,721, FIG. 5D). To examine the SHP1 downstream molecular pathways that are involved in regulating phagolysosome fusion and subsequent cross-presentation, we performed RAB GTPase library mini-screen and identified that overexpression of SHP1 most dramatically decreased RAB34 expression (data not shown; see Yuan et al. 2022. FIG. 5H; immunoblot of Rab GTPase proteins SHP1, RAB4, RAB5, RAB7, RAB9a, RAB11, RAB34 in Ptpn6 overexpressed HEK-293 cells; see also U.S. Provisional Application No. 63/252,721. FIG. 5E), whereas knockdown of SHP1 increased RAB34 expression (data not shown; see Yuan et al. 2022, FIG. 5I; immunoblot of Rab34 and SHP1 proteins in Ptpn6 overexpressed or knockdown HEK-293 cells; see also U.S. Provisional Application No. 63/252,721, FIG. 5F). Furthermore, knockdown SHP1 in mouse BMDCs reduced cytosolic RAB34 but increased membrane accumulation of RAB34 (data not shown; see Yuan et al. 2022, FIG. 5J; Cytosol, membrane Rab34 levels in siCtrl- and siPtpn6-BMDCs; see also U.S. Provisional Application No. 63/252,721, FIG. 5G). VitE-treated BMDCs also had increased total RAB34 expression (data not shown: see Yuan et al. 2022, FIG. 5K; immunoblot of Rab34 proteins in the vehicle (Veh) or VitE treated BMDCs for 48 hr; see also U.S. Provisional Application No. 63/252,721, FIG. 5H), as well as increased membrane RAB34 and decreased cytosolic RAB34 proteins (data not shown; see Yuan et al. 2022, FIG. 5L; Cytosol, membrane Rab34 levels in BMDCs pre-treated with vehicle (Veh) or VitE for 48 hr; see also U.S. Provisional Application No. 63/252,721, FIG. 5I). Moreover, compared with control BMDCs, silencing RAB34 in BMDCs (siRab 34-DCs) reduced the proliferation of antigen-specific OT-1 T cells in coculture (data not shown; see Yuan et al. 2022, FIG. 5M; CFSE-dilution analysis of OT-1 T cells co-cultured with OVA protein-pulsed BMDCs with knockdown of Rab34 by siCtrl or siRab34; see also U.S. Provisional Application No. 63/252,721, FIG. 5J), signifying the decreased cross-presentation efficiencies of siRab 34 DCs. These data indicate that inhibiting SHP1 by VitE or genetic knockdown increases RAB34 membrane accumulation and suppresses phagolysosomal fusion, thereby promoting antigen cross-presentation and T-cell activation.

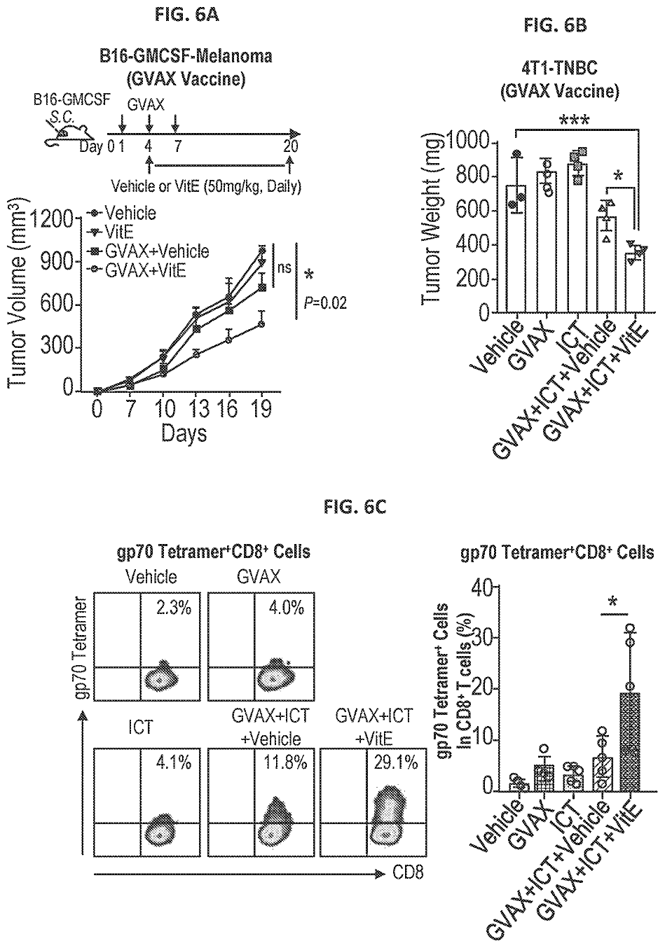

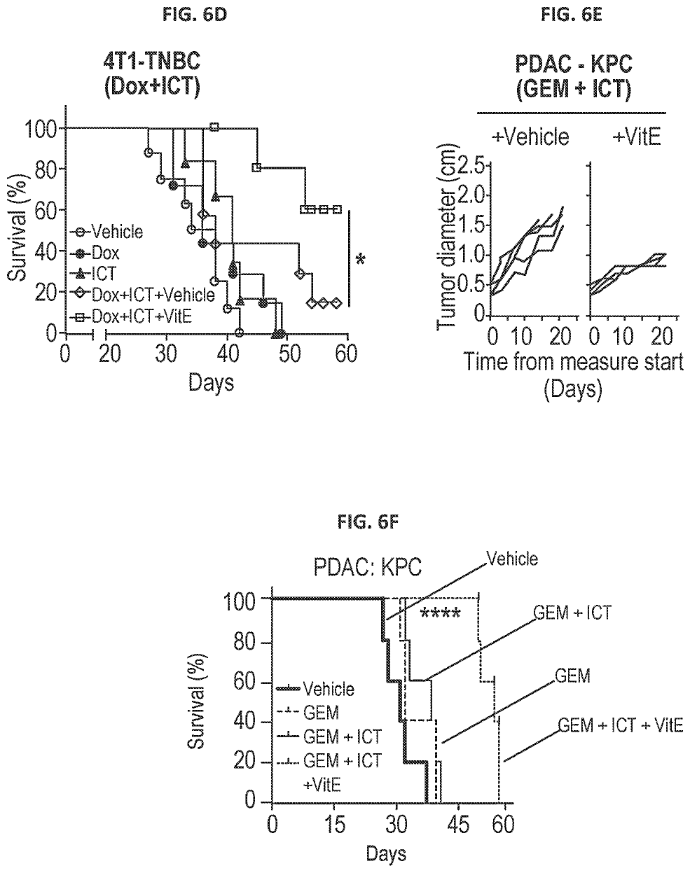

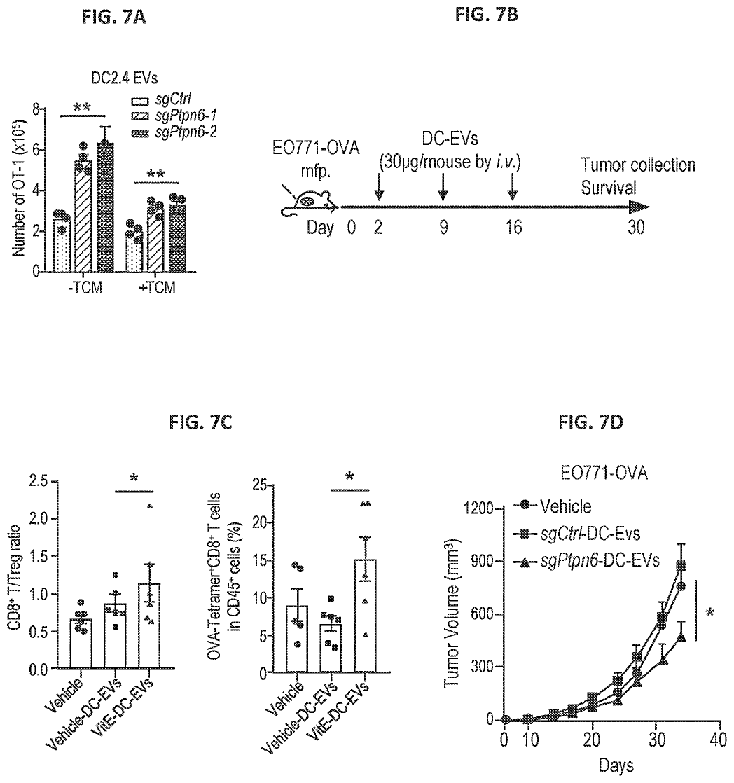

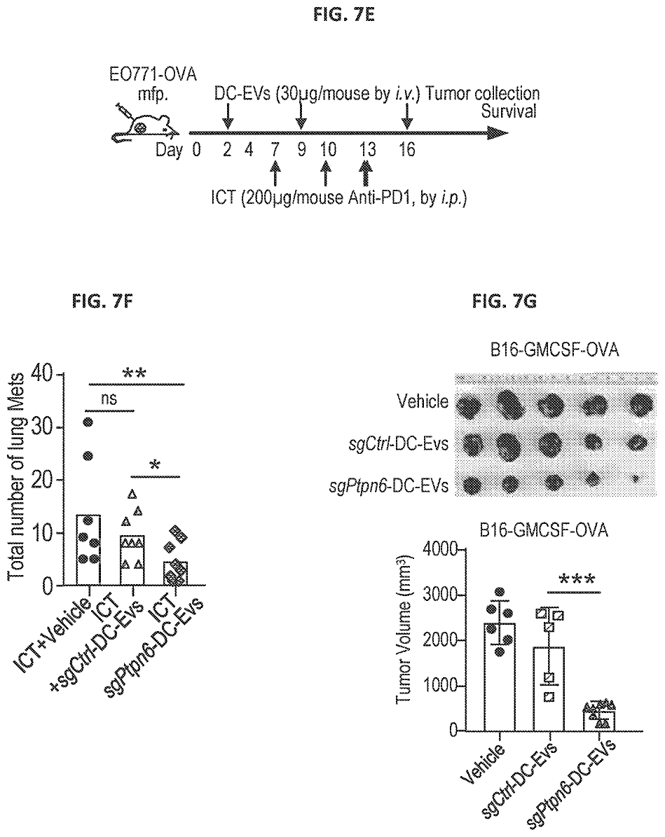

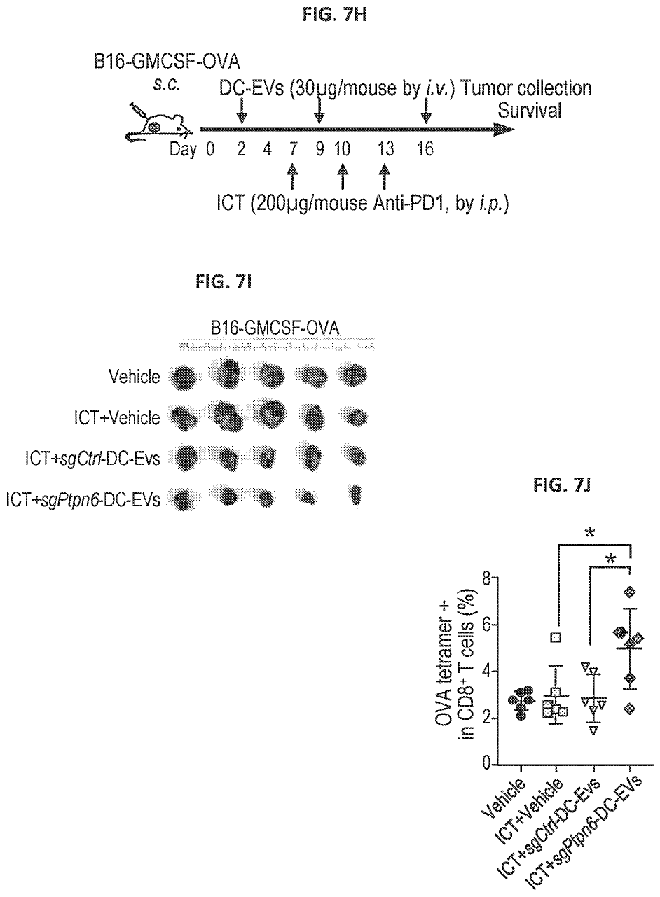

DC-derived extracellular vesicles (DC-EVs) carry biomolecules from DCs including membrane peptide-MHC-I (pMHC-I) complexes which function in antigen cross-presentation as well DCs (Lindenbergh and Stoorvogel, 2018). Remarkably, the VitE-treatment of DCs increased the level of H-2Kb-SIINFEKL complexes on DC-EVs compared to vehicle treatment (FIG. 5A). Confocal microscopy showed that VitE-treated DC-EVs exhibited increased binding to CD8+ T-cells than vehicle-treated DC-EVs (FIG. 5B), and the interaction was peptide-MHC-I complex specific (data not shown; see Yuan et al., FIG. 6C; see also U.S. Provisional Application No. 63/252,721. FIG. 6C). Moreover. compared to vehicle-treated DC-EVs. VitE-treated DC-EVs more effectively enhanced the proliferation and IFN-γ production of T-cells (FIG. 5C). Similarly, EVs from sgPtpn6-DC2.4 also enhanced T-cell proliferation (FIG. 7A), which indicated that EVs from SHP1-knockout DCs have a greater capacity of antigen-presentation relative to the control DC-EVs. These data indicated that EVs from VitE-treated DCs or sgPtpn6-DCs have a greater capacity of antigen-presentation to priming T-cell antitumor immunity.

### Example 6. VitE Augments Immune Induction Therapies

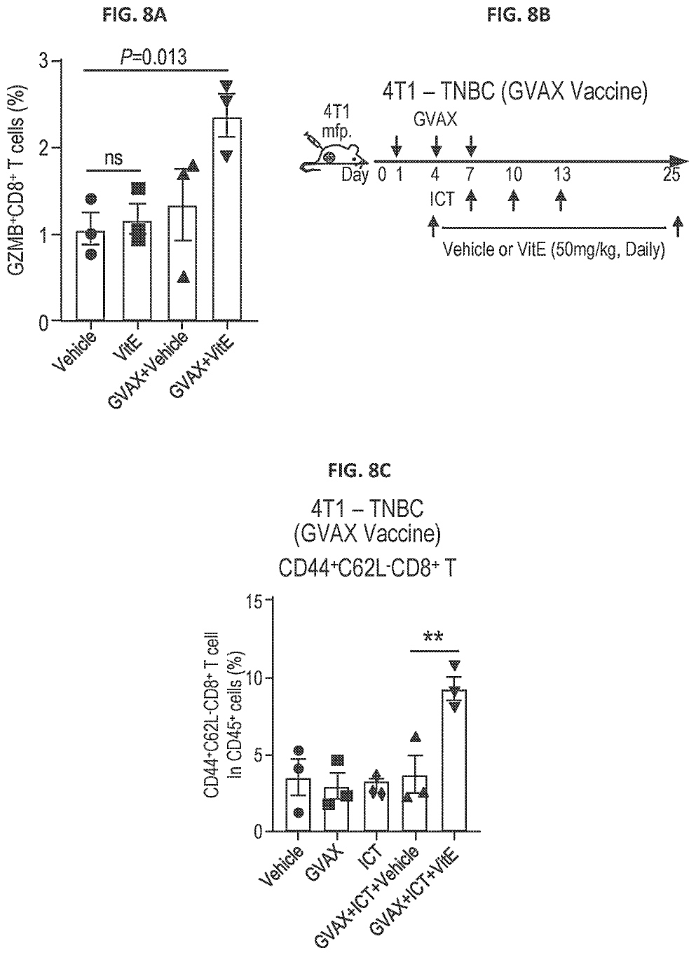

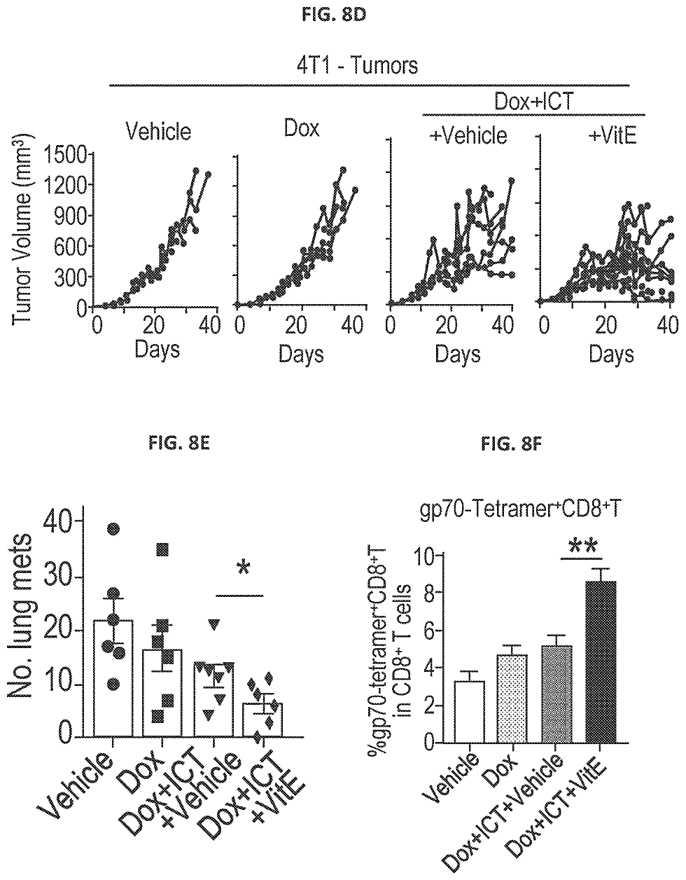

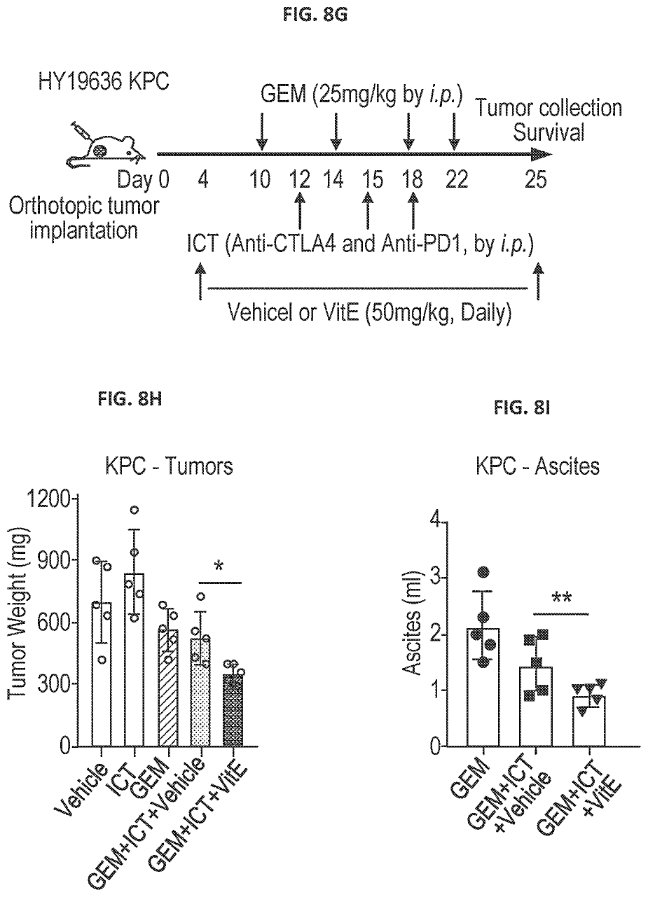

We further tested whether VitE may enhance the efficacies of anticancer therapies, especially, those that release tumor antigens and enrich DCs infiltration, i.e., tumor vaccines or immunogenic chemotherapies. Mice were inoculated with the GM-CSF-secreting tumor cell vaccine (GVAX) post-injection of B16-GMCSF-tumor cells and treated with vehicle or VitE (FIG. 6A). GVAX+VitE led to a significant tumor inhibition (FIG. 6A) and increased tumor-infiltrating cytolytic GZMB CD8⁺ T cells (FIG. 8A). Similarly. VitE treatment combined with GVAX+ICT significantly enhanced the antitumor effect of aggressive 4T1 tumors of a mouse triple-negative breast cancer (TNBC) model (FIG. 6B: FIG. 8B). increased tumor-infiltrating antigen-experienced CD44³⁰ CD62L⁻CD8⁺ T cells (FIG. 8C) and tumor-reactive gp70 tetramer-specific CD8⁺ T cells which recognize the 4TI tumor-associated gp70 antigen (FIG. 6C).

Next, we assessed whether VitE further enhances chemotherapy+ICT antitumor effects in 4T1 tumor-bearing mice treated with low-dose doxorubicin+ICT. Compared to the doxorubicin+ICT group, doxorubicin+ICT+VitE treatment substantially prolonged the survival of mice bearing the ICT-resistant 4T1 tumors (FIG. 6D), inhibited tumor growth and lung metastasis (FIG. 8D-8E). Moreover, adding VitE into the combinatorial treatment (doxorubicin+ICT) increased the infiltration of tumor-infiltrating gp70) tetramer-specific CD8⁺ T cells (FIG. 8F). Pancreatic ductal adenocarcinoma (PDAC) is one of the most aggressive and lethal types of cancer that is resistant to all standard treatments such as gemcitabine (GEM) chemotherapy and immunotherapies. Notably, supplementing VitE to the GEM+ICT combinatorial regimen effectively inhibited the HY19636KPC PDAC tumor growth, reduced tumor-induced ascites, and prolonged the survival time of tumor-bearing mice compared to the GEM+ICT+ vehicle group (FIG. 6E-6F; see Yuan et al. 2022, FIG. 7; FIG. 8G-8I). Collectively, VitE greatly enhanced the antitumor effect of tumor vaccines or immunogenic chemotherapies that release tumor antigens and enrich DC infiltrations, and ultimately sensitized various highly ICT-resistant tumors to the ICT antitumor effect. See Yuan et al, FIG. 7H.

A model of VitE reinvigorating DCs function and boosting anti-cancer immunity is proposed (see Yuan et al. 2022, FIG. 7H; see also U.S. Provisional Application No. 63/252,721, FIG. 7H). A successful anti-cancer immune response requires a series of stepwise events in cancer-immunity cycle, which are critically regulated and can be therapeutically modulated. During tumor development, treatment with immunogenic chemotherapeutics or the use of cancer vaccines release tumor-associated antigens (TAA) to recruit the dendritic cells (DCs) infiltration into the tumor. Functional DCs capture, process, and present tumor antigens to T-cells leading to an antitumor immune response. However, the tumor-associated dendritic cells (TADCs) are mostly dysfunctional with upregulated pSHP1/SHP1 and low levels of cancer-specific peptide-MHC-I (pMHC-I) complexes in DCs or DC-derived extracellular vesicle (DC-EVs), thus TADCs cannot effectively activate cytotoxic T-cells. Upon VitE treatment, VitE enters DCs through the SCARB1 receptor, decreases pSHP1/SHP1, and increases pMHC-I complexes to reactivate TADCs that lead to enhanced cross-presentation of tumor antigens by DCs and DC-EVs resulting in tumor-specific T-cell priming, expansion, and infiltration at the tumor site. When VitE is combined with immune checkpoint therapies (ICT) that reinvigorate and expand anticancer T-cell immune responses, the tumor is effectively inhibited.

### Example 7. VitE-Treated DC-EVs Inhibited Tumor Growth

To investigate if EVs from VitE-treated DCs can trigger tumor rejection in vivo, we injected DC-EVs from vehicle-versus VitE-treated BMDCs loaded with OVA (30 μg) intravenously into mice bearing EO771 mammary tumor cells that express the OVA-antigen (EO771-OVA) weekly for three weeks (FIG. 7B). Slower tumor growth was significantly detected in VitE-DC-EVs-treated mice compared to vehicle-treated DC-EVs and vehicle control groups (FIG. 5D). In addition, VitE-treated DC-EVs induced a significantly higher tumor infiltration of OVA-specific CD8+ T-cell (FIG. 7C), indicating that VitE-treated DC-EVs are potent at eliciting a specific antitumor immune response. Importantly, a prolonged survival rate was achieved in 50% VitE-DC-EVs-treated mice at 60 days after tumor challenge, whereas no mice survived in the vehicle or vehicle-DC-EVs treatment groups (FIG. 5D).

### Example 8. SHP1-Knockout DC-EVs Inhibited Tumor Growth and Empowered ICT Immunotherapy

To test the effect of DC-EVs from SHP1-deficient DCs in antitumor immunity in vivo, we treated E0771-OVA bearing C57BL/6 mice with DC-EVs (30 μg/week×3 weeks by i.v.) from sgCtrl-DC2.4 versus sgPtpn6-DC2.4 cells loaded with OVA (FIG. 7B). Notably, sgPtpn6-DC-EVs treatment significantly inhibited E0771-OVA tumor growth whereas sgCtrl-DC-EVs didn't (FIG. 7D). Moreover, combining sgPtpn6-DC-EVs with ICT (FIG. 7E) yielded a strong therapeutic response in mice bearing the ICT-resistant EO771-OVA tumors, as indicated by delayed tumor growth (FIG. 5E, see also Yuan et al., FIG. S9J), reduced lung metastasis (FIG. 7F), and prolonged survival (FIG. 5E). In contrast, tumor growth curves of EO771-OVA tumor-bearing mice treated with vehicle or ICT (anti-PD1) (n=6) were very similar (data not shown; see U.S. Provisional Application No. 63/252,721, FIG. 8G).

Consistently, DC-EVs were administered intravenously into orthotopic B16-GMCSF-OVA melanoma-bearing mice weekly for three weeks and tumor growth was significantly reduced in sgPtpn6-DC-EVs-treated mice at 25 days after inoculation compared to sgCtrl-DC-EVs and vehicle-treated groups (FIGS. 7G, 5F). Also, compared to ICT alone or sgCtrl-DC-EVs+ICT, sgPtpn6-DC-EVs in combination with ICT (FIG. 7H) markedly increased tumor infiltration of OVA antigen-specific CD8+ T-cells (FIG. 7J) and significantly inhibited tumor growth in mice bearing the B16-GMCSF-OVA melanoma (FIGS. 7I, 5G).

Together, EVs from SHP1-inhibited (by VitE-treatment or Ptpn6-knockout) DCs offer a promising option to generate an antigen-specific immune response and empower immune checkpoint therapy.

### Example 9. The Inhibition of SHP1 in Dendritic Cells (DCS) Delays Tumor Growth in Breast Cancer Brain Metastasis

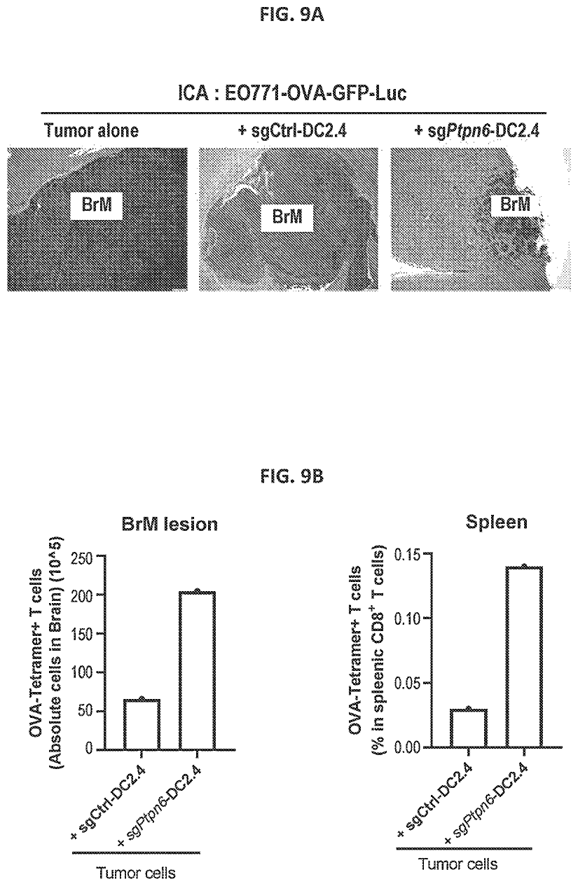

Brain metastases (BrM) represent the last frontier for the treatment of the most common cancers. Unfortunately, there are no curative therapies for patients with BrM, and survival is often measured by only a few months following a BrM diagnosis. To investigate whether SHP1 inhibition in DCs impedes BrM in vivo, the sgCtrl-DC2.4 or sgPtpn6-DC2.4 cells were co-transplanted with EO771-OVA-GFP-Luc cells into breast cancer BrM-bearing mice, the sgCtrl-DC2.4 showed mild antitumor effects, whereas the sgPtpn6-DC2.4 highly inhibited BrM tumor growth (FIG. 9A) and increased the infiltrating tumor-reactive OVA tetramer-specific CD8³⁰ T cells in local BrM lesions and spleens systemically (FIG. 9B). These results indicate that the inhibition of SHP1 in DCs genetically or by pharmaceutical treatment enhances antitumor immunity and delays tumor growth in BrM.

### Example 10. The Pharmacological Inhibition of SHP1 Enhances the Activation of Dendritic Cells (DCs) Derived From Human Induced Pluripotent Stem Cells (iPSCs).

A major obstacle for DC-based immunotherapy is the difficulty to obtain a sufficient number of functional DCs. Conventionally, monocyte derived DCs (moDCs) have been used as antigen presenting cells (APCs) in vitro and in vivo, however the number, quality and antigen presenting ability is donor dependent. Theoretically, this limitation can be overcome by using induced pluripotent stem cells (iPSCs) such as embryonic stem cells (ESCs) and induced pluripotent stem cells (iPSCs) as an unlimited source of DCs.

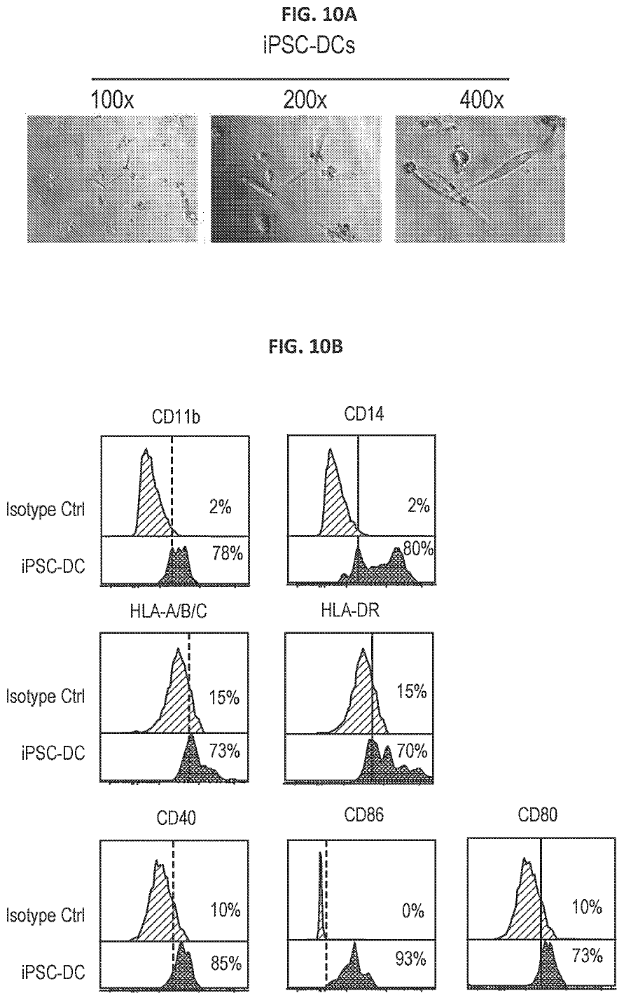

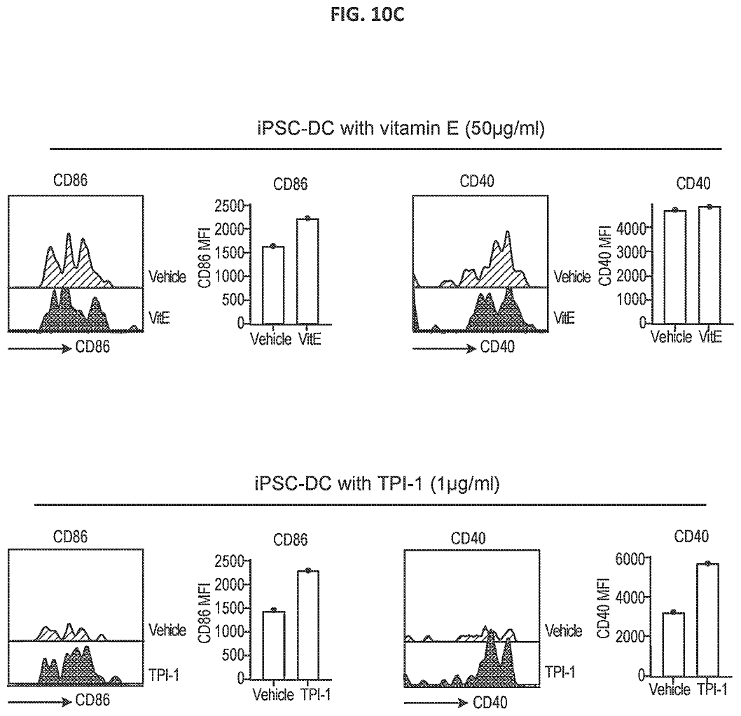

We explored the feasibility of differentiating populations of primary DCs from iPSCs and characterized the phenotype of iPSC-DC with the pharmacological inhibition of SHP1 by vitamin E or TPI-1. Firstly, we differentiated human iPSCs to DCs under the culture conditions with the addition and withdrawal of specific growth factors and cytokines. On microscopic observations, the original iPSC cells were found to be small and round, which enlarged upon differentiation, and further stimulation induced the development of distinct dendrites and cytoplasmic protrusions on day 30 (FIG. 10A). These morphological changes of floating iPSC-derived DC cells were concordant with the positive for CD11c and Major Histocompatibility Complex (MHC) class II (HLD-DR), along with the high expression of classical APC surface molecules such as CD11b, HLA-A/B/C, CD40, CD80, CD86, observed in flow cytometric analysis (FIG. 10B). Notably, the pharmacological inhibition of SHP1 with vitamin E (50 μg/ml) or TPI-1 (1 μg/ml) treatment effectively enhance the activation of human iPSC-DCs, as indicated by the increased expression of DC activation markers CD40 and CD86 (FIG. 10C).

### REFERENCES

1. Abram, C.L., Roberge, G.L., Pao, L.I., Neel, B.G., and Lowell, C.A. (2013). Distinct roles for neutrophils and dendritic cells in inflammation and autoimmunity in motheaten mice. Immunity 38, 489-501.
2. Arnaud-Coffin, P., Maillet, D., Gan, H.K., Stelmes, J.J., You, B., Dalle, S., and Peron, J. (2019). A systematic review of adverse events in randomized trials assessing immune checkpoint inhibitors. International journal of cancer Journal international du cancer 145, 639-648.
3. Carmi, Y., Prestwood, T.R., Spitzer, M.H., Linde, I.L., Chabon, J., Reticker-Flynn, N.E., Bhattacharya, N., Zhang, H., Zhang, X., Basto, P.A., et al. (2016). Akt and SHP1 are DC-intrinsic checkpoints for tumor immunity. JCI insight 1, e89020.
4. Chen, D.S., and Mellman, I. (2013). Oncology meets immunology: the cancer-immunity cycle. Immunity 39, 1-10.
5. Cruz, F.M., Colbert, J.D., Merino, E., Kriegsman, B.A., and Rock, K.L. (2017). The Biology and Underlying Mechanisms of Cross-Presentation of Exogenous Antigens on MHC-I Molecules. Annual review of immunology 35, 149-176.
6. De Henau. O., Rausch, M., Winkler, D., Campesato, L.F., Liu, C., Cymerman, D.H., Budhu, S., Ghosh, A., Pink, M., Tchaicha, J., et al. (2016). Overcoming resistance to checkpoint blockade therapy by targeting PI3Kgamma in myeloid cells. Nature 539, 443-447.
7. Galluzzi, L., Humeau, J., Buque, A., Zitvogel, L., and Kroemer, G. (2020). Immunostimulation with chemotherapy in the era of immune checkpoint inhibitors. Nature reviews Clinical oncology 17, 725-741.
8. Galon, J., and Bruni, D. (2019). Approaches to treat immune hot, altered and cold tumours with combination immunotherapies. Nature reviews Drug discovery 18, 197-218.
9. Guermonprez, P., Valladeau, J., Zitvogel, L., Thery, C., and Amigorena, S. (2002). Antigen presentation and T cell stimulation by dendritic cells. Annual review of immunology 20, 621-667.
10. Hegde, P.S., and Chen, D.S. (2020). Top 10 Challenges in Cancer Immunotherapy. Immunity 52, 17-35.
11. Karsunky, H., Merad, M., Cozzio, A., Weissman, I.L., and Manz, M.G. (2003). Flt3 ligand regulates dendritic cell development from Flt3+lymphoid and myeloid-committed progenitors to Flt3+ dendritic cells in vivo. The Journal of experimental medicine 198, 305-313.
12. Kowal, J., and Tkach, M. (2019). Dendritic cell extracellular vesicles. Int Rev Cell Mol Biol 349, 213-249.
13. Lin, M.L., Zhan, Y., Proietto, A.I., Prato, S., Wu, L., Heath. W.R., Villadangos, J.A., and Lew, A.M. (2008). Selective suicide of cross-presenting CD8+ dendritic cells by cytochrome c injection shows functional heterogeneity within this subset. Proceedings of the National Academy of Sciences of the United States of America 105, 3029-3034.
14. Lindenbergh, M.F.S., and Stoorvogel, W. (2018). Antigen Presentation by Extracellular Vesicles from Professional Antigen-Presenting Cells. Annual review of immunology 36, 435-459.
15. Lorenz, U. (2009). SHP1 and SHP-2 in T cells: two phosphatases functioning at many levels. Immunological reviews 228, 342-359.
16. Ludin, A., and Zon, L.I. (2017). Cancer immunotherapy: The dark side of PD-1 receptor inhibition. Nature 552, 41-42.
17. Marar, C., Starich, B., and Wirtz, D. (2021). Extracellular vesicles in immunomodulation and tumor progression. Nature immunology.
18. Pardoll, D.M. (2012). The blockade of immune checkpoints in cancer immunotherapy. Nature reviews Cancer 12. 252-264.
19. Pitt, J.M., Andre, F., Amigorena, S., Soria, J.C., Eggermont, A., Kroemer, G., and Zitvogel, L. (2016). Dendritic cell-derived exosomes for cancer therapy. The Journal of clinical investigation 126, 1224-1232.
20. Sharma, P., and Allison, J.P. (2015). The future of immune checkpoint therapy. Science 348, 56-61.
21. Sharma, P., Siddiqui, B.A., Anandhan, S., Yadav, S.S., Subudhi, S.K., Gao, J., Goswami, S., and Allison, J.P. (2021). The Next Decade of Immune Checkpoint Therapy. Cancer discovery 11, 838-857.
22. Topalian, S.L., Drake, C.G., and Pardoll, D.M. (2015). Immune checkpoint blockade: a common denominator approach to cancer therapy. Cancer cell 27, 450-461.
23. Wculek, S.K., Cueto, F.J., Mujal, A.M., Melero, I., Krummel, M.F., and Sancho, D. (2020). Dendritic cells in cancer immunology and immunotherapy. Nature reviews Immunology 20, 7-24.
24. Yang, J., Liu, L., He. D., Song, X., Liang, X., Zhao, Z.J., and Zhou, G.W. (2003). Crystal structure of human protein-tyrosine phosphatase SHP1. The Journal of biological chemistry 278, 6516-6520.
25. Zong, J., Keskinov, A.A., Shurin, G.V., and Shurin, M.R. (2016). Tumor-derived factors modulating dendritic cell function. Cancer immunology, immunotherapy: CII 65, 821-833.

The above description of example embodiments of the present disclosure has been presented for the purposes of illustration and description. It is not intended to be exhaustive or to limit the disclosure to the precise form described, and many modifications and variations are possible in light of the teaching above.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

A recitation of “a,” “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The terms “about” and “approximately” as used herein shall generally mean an acceptable degree of error for the quantity measured given the nature or precision of the measurements. Typically. exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X. 0.9X, 0.91X, 0.92X, 0.93X. 0.94X, 0.95X, 0.96X, 0.97X, 0.98X, 0.99X, 1.01X, 1.02X, 1.03X, 1.04X, 1.05X, 1.06X, 1.07X, 1.08X, 1.09X. 1.1X, 1.11X, 1.12X, 1.13X, 1.14X, 1.15X, 1.16X. 1.17X, 1.18X, 1.19X, and 1.2X. Thus, “about X” is intended to teach and provide written description support for a claim limitation of, e.g., “0.98X.”

All patents, patent applications, publications, and descriptions mentioned herein are incorporated by reference in their entirety for all purposes. None is admitted to be prior art. Where a conflict exists between the instant application and a reference provided herein, the instant application shall dominate.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “‘1’ or ‘2’ or ‘3’ or ‘1 and 2’ or ‘1 and 3’ or ‘2 and 3’ or ‘1, 2 and 3’”. Whenever a range is given in the specification, for example, a temperature range, a time range, or a composition range, all intermediate ranges and subranges, as well as all individual values included in the ranges given are intended to be included in the disclosure.

What is claimed is:

1. A method of treating cancer or an infection in a subject, the method comprising administering to the subject dendritic cell (DC)-derived extracellular vesicles (DC-EVs), wherein the DC-EVs are obtained from dendritic cells that have been treated to inhibit src homologous region 2 domain-containing phosphatase-1 (SHP1) in the cells, and wherein the dendritic cells have been loaded with an antigen present in the cancer of the subject or in an infectious agent present in the subject.

2. The method of claim 1, wherein the dendritic cells are loaded by culturing them in the presence of the antigen for a duration sufficient to allow the presentation of the antigen on the surface of the dendritic cells, and wherein the dendritic cells are cultured under conditions permissible for exosome production.

3. The method of claim 1 or 2, further comprising administering the dendritic cells to the subject together with the DC-EVs.

4. The method of claim 1 or 2, wherein the DC-EVs are harvested from the dendritic cells and concentrated prior to administration to the subject.

5. The method of claim 1 or 2, wherein the dendritic cells are monocyte-derived dendritic cells (MoDCs) or induced pluripotent stem cell (iPSC)-derived dendritic cells (iPSC-DCs).

6. The method of claim 1 or 2, wherein SHP1 is inhibited in the dendritic cells by contacting the cells with a small molecule SHP1 inhibitor, by silencing SHP1 in the cells, or by editing the SHP1 gene in the cells so as to reduce or eliminate its expression.

7. The method of claim 6, wherein the small molecule SHP1 inhibitor is vitamin E (DL-α-tocopherol) or an analog or variant thereof.

8. The method of claim 6, wherein the small molecule SHP1 inhibitor comprises the structure

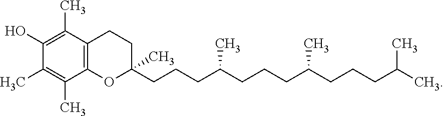 [SMILES: Cc1c(C)c2c(c(C)c1O)CC[C@@](C)(CCC[C@H](C)CCC[C@H](C)CCCC(C)C)O2]

9. The method of claim 6, wherein the small molecule SHP1 inhibitor is at least one of TPI-1, NSC-87877, suramin, or sodium stibogluconate (SSG).

10. The method of claim 6, wherein the SHP1 gene is edited in the cells using a CRISPR-Cas system, comprising a guide RNA that targets the SHP1 locus.

11. The method of claim 1 or 2, wherein the antigen is
(a) a tumor antigen that is expressed on the surface of a tumor cell of the subject's cancer or an antigenically active fragment thereof, or a peptide fragment of the tumor antigen that is capable of being presented by dendritic cells to T cells; or
(b) a viral or bacterial antigen that is expressed on the surface of the infectious agent causing the infection or an antigenically active fragment thereof, or a peptide fragment of the viral or bacterial antigen that is capable of being presented by dendritic cells to T cells.

12. The method of claim 1 or 2, wherein the extracellular vesicles comprise exosomes.

13. The method of claim 1 or 2, wherein inhibiting SHP1 in the dendritic cells and culturing the cells in the presence of the antigen results in an increased amount of the antigen on the surface of the dendritic cells as compared to control dendritic cells that have been cultured in the presence of the antigen but in which SHP1 has not been inhibited.

14. The method of claim 13, wherein the extracellular vesicles harvested from the dendritic cells have an increased amount of the antigen on their surfaces as compared to extracellular vesicles harvested from the control dendritic cells that have been cultured in the presence of the antigen but in which SHP1 has not been inhibited.

15. The method of claim 1 or 2, wherein the subject has cancer and the method further comprises administering an anti-cancer therapy to the subject as combinational therapy, wherein the anti-cancer therapy comprises surgical therapy, chemotherapy, radiation therapy, cryotherapy, hormonal therapy, and/or immunotherapy.

16. The method of claim 15, wherein the anti-cancer therapy comprises administering a tyrosine kinase inhibitor, co-stimulatory mAb, epigenetic modulator, chemotherapeutic agent, radiation therapeutic, vaccine, adoptive T-cell therapeutic, or oncolytic virus to the subject.

17. The method of claim 1 or 2, wherein the subject has a solid tumor or hematological cancer.

18. The method of claim 17, wherein the solid tumor or hematological cancer is melanoma, breast cancer, lung cancer, colorectal cancer, liver cancer, gastric cancer, esophageal cancer, pancreatic cancer, head and neck cancer, ovary cancer, cervical cancer, urothelial cancer, renal cell cancer, bladder cancer, prostate cancer, lymphoma, and/or leukemia.

19. The method of claim 1 or 2, wherein the subject has a viral infection or bacterial infection.

20. The method of claim 1 or 2, wherein the method further comprises administering immunotherapy to the subject.

21. The method of claim 20, wherein the immunotherapy comprises administering an immune checkpoint blockade binding agent to the subject.

22. The method of claim 21, wherein the immune checkpoint blockade binding agent is antibody comprising one or more of an anti-CTLA4 antibody, an anti-PD1 antibody, an anti-PD-L1 antibody, an anti-LAG-3 antibody, an anti-TIM-3 antibody, an anti-TIGIT antibody, an anti-CD47 antibody, or an anti-VISTA antibody.

23. The method of claim 22, wherein the antibody is a human antibody, chimeric antibody, humanized antibody, an F(ab)′2, an Fab, an Fv, a single domain antibody, a bispecific antibody, a helix-stabilized antibody, a single-chain antibody molecule, a disulfide stabilized antibody, or a domain antibody.

24. The method of claim 20, wherein the immunotherapy comprises administering a CAR-T cell, a CAR-NK cell, a CAR-Macrophage, a tumor vaccine, an oncolytic virus vaccine, a vaccine against an infectious agent, co-stimulatory mAb, epigenetic modulator, TLR3/7/8/9 agonist, anti-CD47, and/or IL-2 receptor agonist to the subject.

25. The method of claim 1 or 2, wherein the DC-EVs are present within a pharmaceutical formulation.

26. A method of producing dendritic cells (DC) for use in treating a subject with cancer or with an infection, the method comprising:
(a) providing a plurality of dendritic cells;
(b) treating the plurality of dendritic cells so as to inhibit src homologous region 2 domain-containing phosphatase-1 (SHP1) in the cells; and
(c) culturing the dendritic cells in the presence of an antigen from a tumor or an infectious agent, wherein the dendritic cells are cultured for a duration sufficient to allow the presentation of the antigen or a fragment thereof on the surface of the dendritic cells, and wherein the dendritic cells are cultured under conditions permissible for exosome production.

27. The method of claim 26, further comprising:
(d) harvesting extracellular vesicles from the dendritic cells; and
(e) concentrating the collected dendritic cell-derived extracellular vesicles (DC-EVs).

28. The method of claim 26 or 27, wherein the dendritic cells are monocyte-derived dendritic cells (MoDCs) or induced pluripotent stem cell (iPSC)-derived dendritic cells (iPSC-DCs).

29. The method of claim 26 or 27, wherein the plurality of dendritic cells are treated by contacting the cells with a small molecule SHP1 inhibitor, by silencing SHP1 in the cells, or by editing the SHP1 gene in the cells so as to reduce or eliminate its expression.

30. The method of claim 29, wherein the small molecule SHP1 inhibitor is vitamin E (DL-α-tocopherol) or an analog or variant thereof.

31. The method of claim 29, wherein the small molecule SHP1 inhibitor comprises the structure

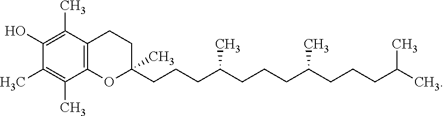 [SMILES: Cc1c(C)c2c(c(C)c1O)CC[C@@](C)(CCC[C@H](C)CCC[C@H](C)CCCC(C)C)O2]

32. The method of claim 29, wherein the small molecule SHP1 inhibitor is TPI-1, NSC-87877, suramin, or sodium stibogluconate (SSG).

33. The method of claim 29, wherein the SHP1 gene is edited in the cells using a CRISPR-Cas system, comprising a guide RNA that targets the SHP1 locus.

34. The method of claim 26 or 27, wherein the antigen is
(a) a tumor antigen that is expressed on the surface of a tumor cell of the subject's cancer or an antigenically active fragment thereof, or a peptide fragment of the tumor antigen that is capable of being presented by dendritic cells to T cells; or
(b) an antigen that is expressed on the surface of the infectious agent causing the infection or an antigenically active fragment thereof, or a peptide fragment of the viral or bacterial antigen that is capable of being presented by dendritic cells to T cells.

35. A dendritic cell produced using the method of any one of claims 26 to 34.

36. Extracellular vesicles harvested from the dendritic cell of any one of claims 26 to 34.

37. A pharmaceutical formulation comprising the dendritic cell of claim 35 or extracellular vesicles of claim 36.
